(19) United States
(12) Patent Application Publication
(10) Pub. No.: US 2023/0039987 A1
(43) Pub. Date: Feb. 9, 2023

(54) GEL COMPOSITIONS, SYSTEMS, AND METHODS

(71) Applicants: Boston Scientific Scimed, Inc., Maple Grove, MN (US); Trustees of Boston University, Boston, MA (US)

(72) Inventors: Lauren S. Lydecker, Millbury, MA (US); Gerald Fredrickson, Westford, MA (US); Samantha Berry, Brighton, MA (US); Katherine A. Cook, Allston, MA (US); Mark Grinstaff, Brookline, MA (US)

(73) Assignees: Boston Scientific Scimed, Inc., Maple Grove, MN (US); Trustees of Boston University, Boston, MA (US)

(21) Appl. No.: 17/868,656

(22) Filed: Jul. 19, 2022

Related U.S. Application Data
(60) Provisional application No. 63/223,808, filed on Jul. 20, 2021.
(60) Provisional application No. 63/260,113, filed on Aug. 10, 2021.

(51) Int. Cl. A61K 31/75; A61K 31/785; A61K 31/795; A61K 31/765; A61K 9/06

(52) U.S. Cl. CPC A61K 31/75; A61K 31/785; A61K 31/795; A61K 31/765; A61K 9/06

(57) ABSTRACT

Methods of forming a gel and related methods of treating subjects with such gels are described. The method may include preparing a composition by combining a macromer comprising a first polyethylene glycol (PEG)-based polymer, a poly(ethylenimine)-based polymer, or a poly(1,2-glycerol) carbonate-based polymer, the macromer including at least one first functional moiety, a crosslinking agent comprising a second PEG-based polymer that includes at least one second functional moiety, and a photoinitiator, and activating the photoinitiator via a light source to form the gel.

### CROSS-REFERENCE TO RELATED APPLICATIONS

This application claims priority to U.S. Provisional Application No. 63/223,808 filed on Jul. 20, 2021, and to U.S. Provisional Application No. 63/260,113 filed on Aug. 10, 2021, both of which are incorporated by reference herein in their entireties.

### TECHNICAL FIELD

This disclosure relates generally to therapeutic gels useful for medical procedures, including endoscopic procedures. For example, the disclosure includes gels, and compositions and systems formulated to form a gel, e.g., for application to bodily tissue, such as, e.g., the gastrointestinal tract.

### BACKGROUND

Endoscopic procedures, such as endomucosal resection (EMR), endosubmucosal dissection (ESD), and anastomosis, and health conditions such as intentional or disease-originated creation of a fistula, inflammatory bowel disease (IBD), and IBD subsidiary diseases, may result in and/or contribute to damage to tissues of the gastrointestinal (GI) tract. Colorectal cancer is among the leading causes of cancer death in the developed countries. Standard preventative care for patients over 50 years old involves a colonoscopy to biopsy polyps, known as a polypectomy, to assess for colorectal cancer. Practically, a physician inserts an endoscope into the patient's colon while under anesthesia, examines the colon, and then removes the polyps. After removal, the wound is either left open to the internal colon environment or thermally sealed using electrocoagulation. Open wounds after a polypectomy or other endoscopic procedures in the GI tract can result in bleeding, hemorrhaging, and sepsis. Electrocoagulation can result in other complications such as perfusion or post polypectomy coagulation syndrome.

These types of medical procedures and health conditions may leave relatively thin tissue layers of the GI tract wall. Currently, physicians often rely on time or surgical procedures, including clipping or endoscopic suturing to allow healing of the GI tract wall. However, these practices may be unsuitable in certain cases, such as large defects, and/or friable or fibrotic tissue. Complications that may arise include perforation, infection, and sepsis.

### SUMMARY

Methods of forming gels useful in medical procedures are disclosed. The present disclosure includes, for example, a method for forming a gel comprising preparing a composition by combining a macromer comprising a first polyethylene glycol (PEG)-based polymer, a poly(ethylenimine)-based polymer, or a poly(1,2-glycerol) carbonate-based polymer, the macromer including at least one first functional moiety; a crosslinking agent comprising a second PEG-based polymer that includes at least one second functional moiety; and a photoinitiator; and activating the photoinitiator via a light source to form the gel. The gel may be biocompatible and/or biodegradable. The at least one first functional moiety may comprise a thiol group, a vinyl group, an allyl group, an acrylate group, or a norbornene group, for example, and/or the at least one second functional moiety may comprise a thiol group, a vinyl group, an allyl group, an acrylate group, or a norbornene group, the at least one first functional moiety being different from the at least one second functional moiety. In at least one example, the at least one first functional moiety or the at least one second functional moiety may comprise a vinyl group, an allyl group, an acrylate group, or a norbornene group, and the other of the at least one first functional moiety or the at least one second functional moiety may comprise a thiol group. According to some examples herein, the macromer, the crosslinking agent, and the photoinitiator may represent a total of 10-25 wt % of the composition, in relation to a total weight of the composition. Optionally, the molar ratio between the at least one first functional moiety and the at least one second functional moiety may range from 1:1 to 2:1. Additionally or alternatively, the macromer may represent a total of 5-15 wt % of the composition, in relation to a total weight of the composition. The crosslinking agent may represent a total of 5-10 wt % of the composition, in relation to a total weight of the composition. The concentration of the photoinitiator within the composition may range from about 0.1 mM to about 100 mM. In some examples, the crosslinking agent comprises N-hydroxysuccinimide groups and/or maleimide groups. Additionally or alternatively, the macromer may comprise a hyperbranched polymer.

According to some aspects herein, the composition may further comprise a physiological buffer. The light source may emit UV light or visible light. For example, the gel may be formed within five seconds when illuminated with UV light. In some examples, the gel may be formed within ten seconds when the photoinitiator is activated with visible light. Optionally, the composition may further comprise an additive to expedite a gelation time of the composition, the additive comprising a tyrosine derivative. In some aspects, the composition may comprise up to 10 mM of the additive. The tyrosine derivative may comprise, for example, tyrosine methyl ester or tyrosine ethyl ester.

The gels described above and elsewhere herein may be used to treat tissue of a subject, e.g., a human subject. For example, the gels may be used to treat tissue of a gastrointestinal tract of a subject.

The present disclosure also includes a method forming a gel comprising preparing a first solution by combining a macromer comprising a polyethylene glycol (PEG)-based polymer, a poly(ethylenimine)-based polymer, or a poly(1,2-glycerol) carbonate-based polymer, the macromer comprising at least one first functional group, and a first buffer; preparing a second solution by combining: a crosslinking agent comprising a second (PEG)-based polymer that comprises a plurality of second functional groups, and a second buffer, the second buffer having a lower pH than the first buffer; and mixing the first solution with the second solution to form the gel. The gel may be biocompatible and/or biodegradable. The at least one first functional group may comprise a thiol group or an amine group, for example, and/or the plurality of second functional groups may comprise N-hydroxysuccinimide groups or maleimide groups. In some examples, the molecular weight of the macromer may be approximately 2,000 Da. Additionally or alternatively, the molecular weight of the crosslinking agent may be approximately 3,400 Da. In some examples, the molar ratio of the crosslinking agent to the macromer may range from 3:2 to 7:3.

As mentioned above, the gels disclosed herein may be used to treat tissue of a subject. For example, the method of forming a gel may include treating a subject by forming a gel on tissue of a gastrointestinal tract of the subject. In at least one example, the method comprises applying to the tissue a first solution comprising a macromer comprising a polyethylene glycol (PEG)-based polymer, a poly(ethylenimine)-based polymer, or a poly(1,2-glycerol) carbonate-based polymer, the macromer comprising at least one first functional group, and a first buffer; and applying to the tissue a second solution comprising a crosslinking agent comprising a second (PEG)-based polymer that comprises a plurality of second functional groups, and a second buffer, the second buffer having a lower pH than the first buffer; wherein the first solution contacts the second solution to form the gel on the tissue. The first solution may be applied to the tissue before, after, or at the same time as the second solution.

The present disclosure also includes a composition comprising a macromer comprising a polyethylene glycol (PEG)-based polymer, a poly(ethylenimine)-based polymer, or a poly(1,2-glycerol) carbonate-based polymer, wherein the macromer comprises at least one thiol group or amine group; and a crosslinking agent comprising a PEG-based polymer that includes a N-hydroxysuccinimide functional group, a maleimide functional group, or both; wherein the composition is formulated as a hydrogel. The hydrogel may have a gel strength of at least 2,000 Pa and/or a shear force between 0.03-0.90 N/cm² when adhered to a bodily lumen. Additionally or alternatively, the hydrogel may be formulated to withstand a burst pressure of up to approximately 150 mbar when the hydrogel is adhered to colon tissue to fill an aperture in the tissue of about 1 mm by about 5 mm.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIGS. 1A and 1B show exemplary macromer structures according to some aspects of the present disclosure.

FIGS. 2A-2G show exemplary macromers according to some aspects of the present disclosure.

FIGS. 3A and 3B show exemplary crosslinking agent structures according to some aspects of the present disclosure.

FIGS. 4A-4D show exemplary crosslinking agents according to some aspects of the present disclosure.

FIG. 5 is a schematic for formation of a gel according to some aspects of the present disclosure.

FIG. 6 is a schematic for dissolution of a gel according to some aspects of the present disclosure.

FIG. 7 illustrates a mechanism for dissolution of a gel in the presence of cysteine.

FIG. 8 illustrates a mechanism for dissolution of a gel in the presence of water.

FIG. 9 illustrates possible reactions with an exemplary crosslinking agent, according to some aspects of the present disclosure.

FIG. 10 is a chart of gel strength as discussed in Example 1.

FIGS. 11A and 11B are charts of gel strength and gel swelling ratio as discussed in Example 2.

FIGS. 12A-12E are charts of gel strength and gelation time as discussed in Example 3.

FIG. 13 illustrates synthesis of an exemplary crosslinking agent, as discussed in Example 4.

FIG. 14 shows crosslinking agents and macromers used to form gels, as discussed in Example 6.

FIG. 15 shows gelation measurements as discussed in Example 6.

FIGS. 16 and 17 show rheology measurements for hydrogels as discussed in Example 6.

FIG. 18 reports amidation kinetics at the NHS ester and internal ester linkages as discussed in Example 6.

FIG. 19 shows ¹H NMR data to monitor hydrolysis as discussed in Example 6.

FIG. 20 shows storage modulus data for hydrogels at different temperatures, as discussed in Example 6.

FIG. 21 reports swelling of hydrogels, as discussed in Example 6.

FIG. 22 shows adhesion measurements of hydrogels, as discussed in Example 6.

FIG. 23 reports cytotoxicity results for hydrogels, as discussed in Example 6.

FIGS. 24 and 25 report bacterial migration studies for hydrogels, as discussed in Example 6.

FIG. 26 is an SEM image of a hydrogel, as discussed in Example 6.

FIGS. 27 and 28 show agar plate assay results for hydrogels, as discussed in Example 6.

FIG. 29 illustrates synthesis of several exemplary crosslinking agents, as discussed in Example 7.

FIG. 30 shows characteristics measured for various hydrogels, as discussed in Example 8.

FIG. 31 shows SEM images for various hydrogels, as discussed in Example 8.

FIG. 32 shows ¹H NMR spectra for a crosslinking agent before and after reaction with a macromer, as discussed in Example 8.

FIG. 33 shows ¹H NMR spectra investigating NHS-hydrolysis of a crosslinking agent, as discussed in Example 8.

FIG. 34 reports kinetic studies for various hydrogels, as discussed in Example 8.

FIG. 35 reports strain sweep and frequency sweep of a hydrogel as discussed in Example 8.

FIGS. 36 and 37 report storage modulus of hydrogels as discussed in Example 8.

FIG. 38 reports swelling of hydrogels as discussed in Example 8.

FIG. 39 reports dissolution of hydrogels as discussed in Example 8.

FIGS. 40 and 41 report rheological measurements on hydrogels as discussed in Example 8.

FIG. 42 reports dissolution results for hydrogels, as discussed in Example 8.

FIG. 43 reports cell viability of hydrogels as discussed in Example 8.

FIG. 44 shows the in vivo study design as discussed in Example 8.

FIGS. 45-49 show H & E staining of various tissue samples, as discussed in Example 8.

FIG. 50 is a schematic for dissolution of hydrogel used as a wound dressing, as discussed in Example 8.

FIG. 51 illustrates synthesis of several exemplary crosslinking agents, as discussed in Example 9.

FIG. 52 illustrates synthesis of an exemplary macromer, as discussed in Example 10.

FIG. 53 shows results of a TNBS assay detecting primary amines on PEI and PEI-SH molecules as discussed in Example 10.

FIG. 54 shows rheological measurements for hydrogels as discussed in Example 11.

FIG. 55 shows ¹H NMR spectra for crosslinking agents, as discussed in Example 11.

FIG. 56 shows a system used to measure burst pressure, as discussed in Example 11.

FIG. 57 reports burst pressure data for hydrogels as discussed in Example 11.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic. All ranges are understood to include endpoints, e.g., a macromer content between 5 wt % and 10 wt % includes 5 wt %, 10 wt %, and all values between.

Embodiments of this disclosure may address one or more limitations in the art. The scope of the disclosure, however, is defined by the attached claims and not the ability to solve a specific problem. The disclosure includes compositions and systems formulated to form a gel, e.g., hydrogel, and compositions in gel/hydrogel form, e.g., useful for application to tissues of the gastrointestinal tract. The hydrogels herein may serve as a temporary, minimally invasive, in situ hydrogel dressing, applied immediately after the time of a medical procedure such as a polypectomy. The hydrogel may prevent or reduce the likelihood of complications by covering and protecting a wound. From a biomaterials design perspective, the dressing may achieve one or more of the following: 1) form rapidly in situ; 2) adhere to colon tissue; 3) be non-cytotoxic; 4) naturally dissolve over 3-5 days; 5) swell up to 200% to absorb wound exudate; 6) prevent the spread or migration of bacteria; and/or 7) conform to the malleable shape of a colon lumen. The gels herein may be formulated with desired characteristics such as gelation rate, adhesion strength, swelling, cytotoxicity, and/or degradation, as a function of hydrogel composition. The compositions herein may be delivered to a subject by a suitable medical device such as a catheter inserted through an endoscope. For example, a dual lumen catheter may be used. Barrier properties of the hydrogel may help to prevent bacterial migration.

The compositions, systems, and methods herein may offer a range of properties, including among others, inherent cohesion and adhesion to tissue. With such properties, the gels herein may function as a protective barrier to thin, damaged, and/or otherwise compromised tissue of a bodily lumen, e.g., the GI tract. For example, an exemplary composition, e.g., a formulation or system for forming a gel, may be applied to a targeted site along the GI tract and the composition may crosslink to form the gel, which may provide barrier protection/therapy to the targeted site. Components of the compositions and gel systems herein may provide desired properties advantageous for tissue protection, e.g., before, during, and/or after a medical procedure. The compositions and systems herein may be delivered to a targeted site by a suitable method or technique. The properties of the compositions such as, e.g., viscosity, may facilitate the deliverability of the gel-forming formulations to targeted sites via suitable medical devices such as, e.g., single/multi lumen catheters, including endoscopes, and syringes, among other devices useful for medical procedures. For example, the compositions herein in gel form may have a viscosity ranging from about 0.010 Pa*s, e.g. to about 0.015 Pa*s, e.g., approximately 0.013 Pa*s at room temperature (about 22-25° C.). Components of the composition or gel system may crosslink to form the gel, which may include activating one or more components with or in the presence of a stimulus, such as pH value or light. The hydrogels herein may be hydrophilic, three-dimensional polymeric networks formed from a macromer and a crosslinking agent (alternatively referred to herein as a crosslinker). The gels, e.g., hydrogels, herein may be formed by combining a macromer with a crosslinking agent under suitable pH conditions or light exposure to initiate crosslinking.

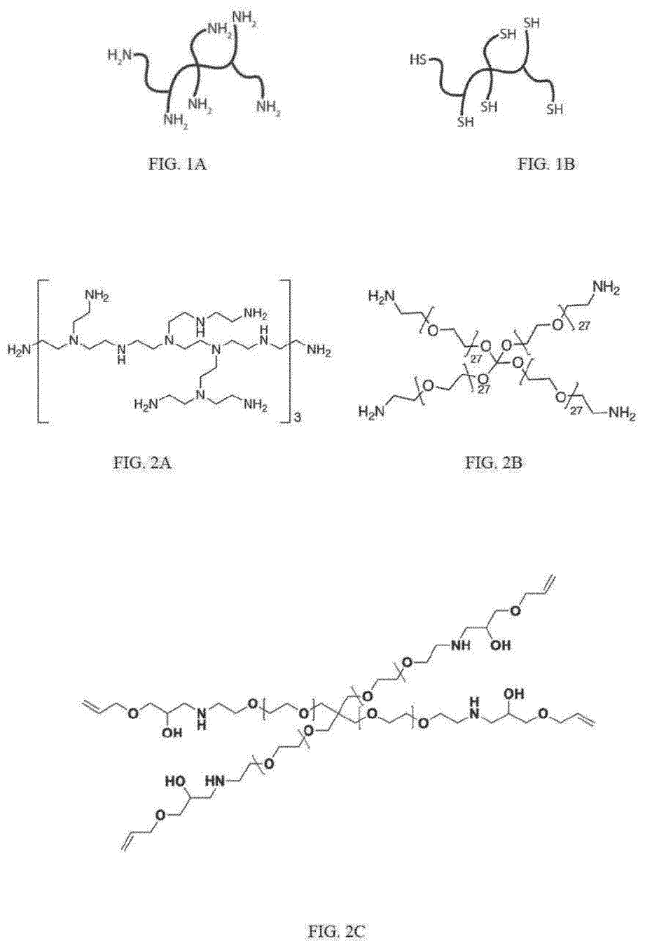

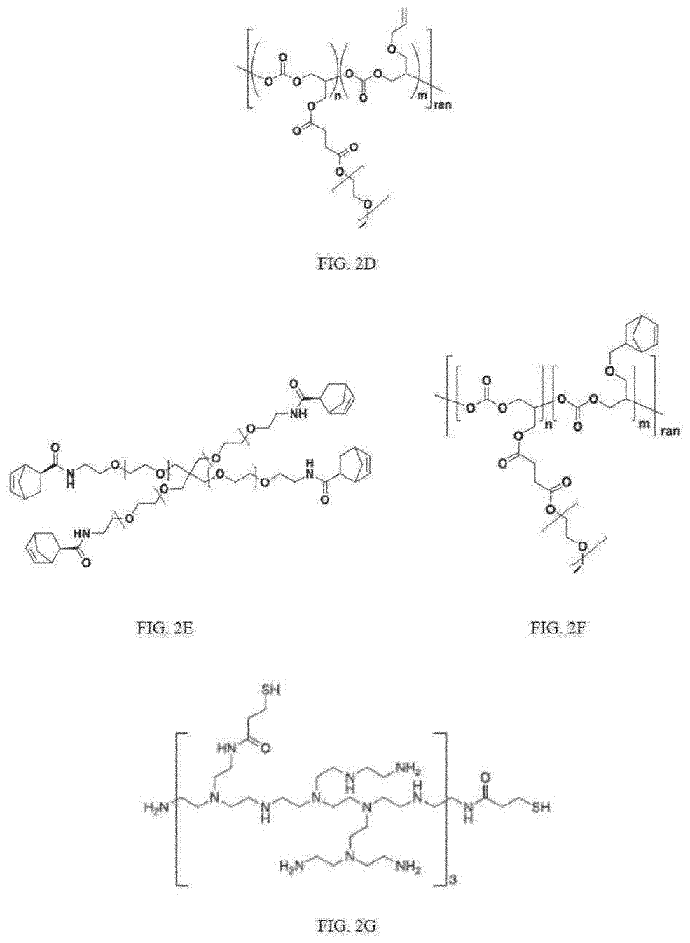

Exemplary macromers useful for the present disclosure include polyethylene glycol (PEG)-based polymers, poly(1,2-glycerol) carbonate (PGC)-based polymers, and poly(ethylene imine)-based polymers. The macromer may have a plurality of functional groups such as amine, alkene, and/or thiol functional groups, available to react with the crosslinking agent. FIGS. 1A and 1B show exemplary macromer structures representing a branched poly(ethylenimine) with amine functional groups (FIG. 1A) and a branched poly(ethylenimine) with thiol functional groups (FIG. 1B). Further examples of macromers that may be used herein are shown in FIG. 2A (poly(ethyleneimine)), FIG. 2B (4-arm PEG-NH₂), FIG. 2C (PEG-based macromer with alkene functional groups), FIG. 2D (poly(1,2-glycerol) carbonate-based macromer with alkene functional group, wherein m and n are integers that represent the amounts of each unit up to a total 100%, and “ran” refers to random copolymer), FIG. 2E (PEG-based macromer comprising a norbornene moiety with alkene functional group), FIG. 2F ((poly(1,2-glycerol carbonate-based macromer comprising a norbornene moiety with alkene functional group, wherein 1, m, and n are integers of 1 or greater), and FIG. 2G (hyperbranched poly(ethyleneimine)-thiol) (see also Example 7). In at least one example, the macromer comprises a poly(1,2-glycerol) carbonate-based polymer with at least one norborene group, wherein the norborene group(s) make up between 1% and 90% of the macromer.

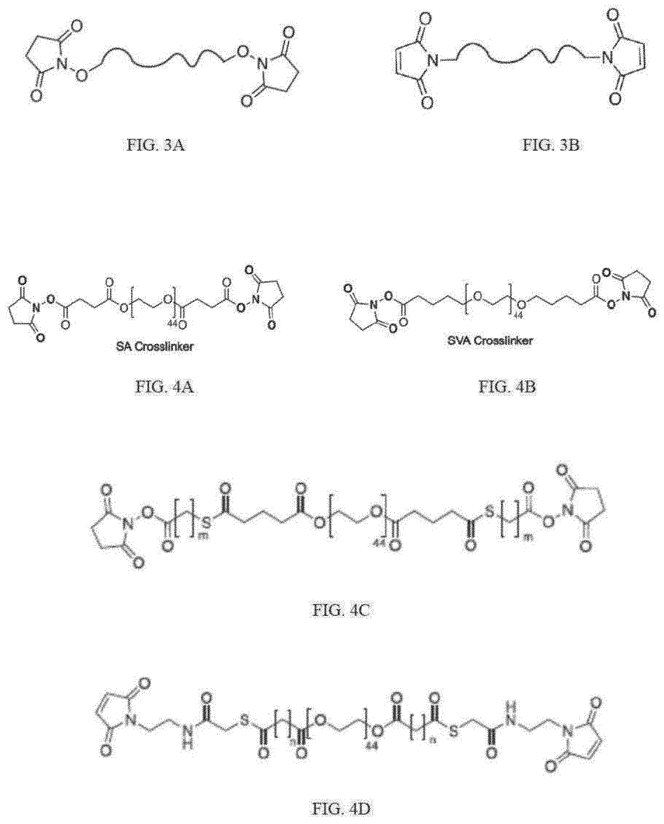

Exemplary crosslinking agents useful for the present disclosure include PEG-based polymers that comprise one or more N-hydroxysuccinimide or maleimide functional groups. FIGS. 3A and 3B show exemplary crosslinking agent structures representing a N-hydroxysuccinimide-PEG polymer (FIG. 3A) and a maleimide-PEG polymer (FIG. 3B). Further examples of crosslinking agents that may be used herein are shown in FIGS. 4A-4D. FIGS. 4A and 4B are two different types of N-hydroxysuccinimide functionalized PEG crosslinking agents. The structures are similar except the one shown in FIG. 4A contains a hydrolysable internal ester linkage. FIG. 4C shows another exemplary structure of N-hydroxysuccinimide functionalized PEG crosslinking agents, wherein m is an integer of 1 or greater, e.g., m=1, 2, 3, 4, 5, 6, 7, 8, 9, 10, 11, 12, 13, 14, 15, 16, 17, 18, 19, or 20 (see also Example 5). FIG. 4D shows an exemplary structure of maleimide functionalized PEG crosslinking agent, wherein n is an integer of 1 or greater, e.g., n=1, 2, 3, 4, 5, 6, 7, 8, 9, 10, 11, 12, 13, 14, 15, 16, 17, 18, 19, or 20 (see also Example 6).

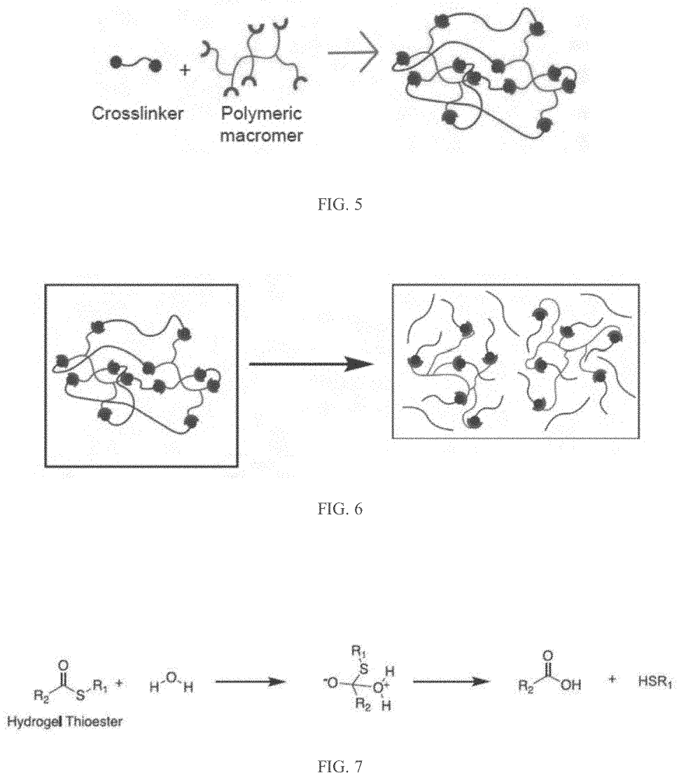

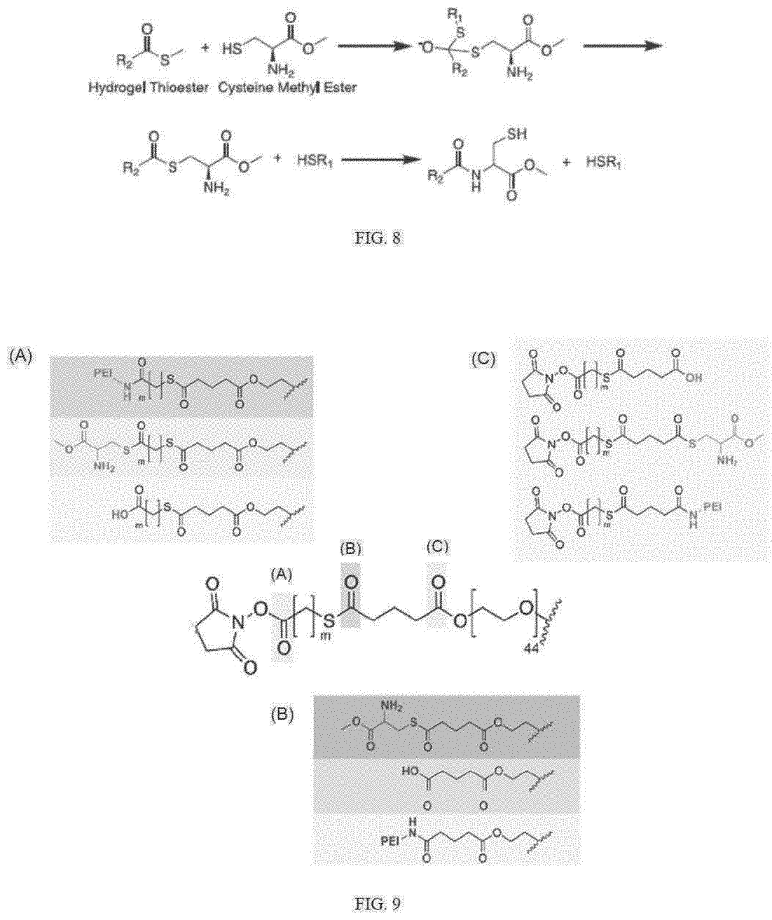

As mentioned above, the gels herein may form three-dimensional polymeric networks capable of forming a barrier over a wound or other site of interest in the body, e.g., in the colon or another portion of the GI tract. FIG. 5 is a simplified illustration of crosslinking between the macromer and the crosslinking agent, wherein functional groups of the macromer react with functional groups of the crosslinking agent. The polymeric network of the gel may be disrupted, e.g., to dissolve the gel, depending on the strength of the bonds between the macromer and crosslinking agent. FIG. 6 is a simplified illustration of dissolution of a gel. Dissolution may occur, for example, through hydrolysis. FIG. 7 illustrates hydrolysis for a hydrogel with thiol groups. Dissolution may also occur via thiol-thioester exchange, such as by reaction with a cysteine methyl ester (FIG. 8) as discussed in several examples below. In the case of the latter, the primary amine on the cysteine methyl ester is believed to rearrange to form an irreversible amide bond, preventing reformation of the gel after the polymeric network disassembles.

The structure of the crosslinking agents may help to control the rate of dissolution. FIG. 9 shows possible reactions at different sites for an exemplary N-hydroxysuccinimide functionalized PEG crosslinking agent: (A) reaction with the NHS ester, (B), reaction with an internal thioester, and (C) reaction with an internal ester. Reactions of these sites with the macromer poly(ethylenimine), a cysteine methyl ester, and water are shown, where darker shaded regions correspond with greater reactivity. Thus, the NHS ester (A) is most reactive with the macromer poly(ethylenimine), and the internal thioester (B) is most reactive with the cysteine methyl ester. The internal ester has comparable reactivity for the macromer, the cysteine methyl ester, and water. Without being bound by theory, it is believed that stability of the gel may derive at least in part to the hydrophobic methylene chain length protecting the adjacent thioester from hydrolysis or thiol-thioester exchange.

The gels herein may be formed on target tissue of a subject, such as tissue of the GI tract (e.g., intestinal tissue, colon tissue, etc.). For example, a crosslinking agent and a macromer may be delivered separately to a target tissue site, such that the two components do not contact each other until they reach the target tissue site. In some examples, a dual lumen catheter may be used, e.g., the crosslinking agent and the macromer being delivered to the target tissue site in separate lumens. The two components may come into contact with each other at the target tissue site, wherein gelation occurs due to a suitable pH (e.g., the components being formulated to crosslink at physiological pH of the GI tract) or in the presence of a photoinitiator activated by UV or visible light at the target tissue site. For example, a photoinitiator may be applied to the target tissue site before, after, or at the same time as the crosslinking agent and/or macromer, and light applied thereafter to activate the photoinitiator and initiate crosslinking to form the gel. Gelation may be initiated in a time greater than 0 and less than 30 seconds, less than 25 seconds, less than 20 seconds, less than 15 seconds, less than 10 seconds, or less than 5 seconds, e.g., a period of time greater than 1 second and less than 15 seconds. The crosslinking agent and macromer (and photoinitiator, when present) may be selected to provide relatively fast gelation kinetics to form a gel in tortuous environments such as the GI tracts and when subjected to the pull of gravity.

Once formed on tissue in situ, the gel may form a barrier with sufficient strength to remain intact for a desired period of time. For example, the gel may remain on the tissue for at least 1 hour, at least 2 hours, at least 4 hours, at least 6 hours, at least 12 hours, at least 16 hours, at least 20 hours, at least 24 hours, at least 2 days, at least 5 days, at least 7 days, at least 14 days, at least 21 days or at least 30 days. According to some aspects of the present disclosure, the gel may form a barrier on tissue that remains for a period of time ranging from about 1 hour to about 60 days, about 1 hour to about 30 days, about 1 hour to about 14 days, about 1 hour to about 24 hours, about 12 hours to about 48 hours, or about 2 days to about 21 days, about 5 days to about 14 days.

The crosslinking agent and the macromer may be selected to provide sufficient strength to suit the period of time desired for the gel to form a barrier on tissue. As discussed in the examples below, greater crosslinking density (at least partially dependent on the number and type of functional groups of the crosslinking agent and the macromer) and/or relative hydrophobicity is expected to provide for stronger gels with longer residence times when applied to tissue. The gels herein may be biocompatible and/or biodegradable. For example, the gels may dissolve over time (e.g., by hydrolysis and/or in the presence of an external thioester such as by thiol-thioester exchange as discussed in the examples below), depending on crosslinking density and strength of the gel.

Below is further discussion of exemplary compositions and systems useful for medical procedures including endoscopic procedures, e.g., the compositions or systems comprising a gel or being formulated to form a gel. The compositions may be applied to a subject for treatment purposes, and the composition may be activated (e.g., to form a gel on-site) via different mechanisms.

pH-Activated Gel Systems

In some aspects of the present disclosure, the composition or system may be formulated to crosslink and form an adhesive, cohesive gel under physiological pH, e.g., a pH of about 7 to about 7.5, such as about 7.35-7.45. Thus, such compositions and systems may be pH-activated such that the gel, e.g., hydrogel, selectively crosslinks at a neutral to basic pH (e.g., little to no crosslinking at acidic pH), with reaction kinetics increasing as the pH increases. According to some aspects of the present disclosure, the composition or system may be pH-activated in order to from a gel, e.g., a hydrogel. For example, the composition may comprise at least two components, e.g., a first component (e.g., a first part solution) and a second component (e.g., a second part solution), that crosslink at a physiological pH, e.g., within a range of about 7 to about 7.5. Thus, for example, the first and second part solutions may have different pH values and may form a gel when mixed together so as to provide for the physiological pH.

A first component of an exemplary system may include a macromer. The macromer may be a multi-functional polyethylene glycol (PEG)-based or poly(ethylene imine)-based polymer. For example, the PEG-based polymer or poly(ethylenimine)-based polymer may have a molecular weight of at least 1500 Da (g/mol), such as about 1800 Da to about 2200 Da, e.g., about 2000 Da. For example, the macromer may have a molecular weight ranging from about 1500 Da to about 2500 Da, from about 1500 Da to about 2000 Da, or from about 1800 Da to about 2200 Da. The poly(ethylenimine) may be linear or branched. In some examples, the macromer may be a multi-functional PEG-based or poly(ethylenimine)-based polymer having a plurality of functional groups. The plurality of functional groups may react with a crosslinking agent of the system (examples of crosslinking agents discussed in further detail below). Such functional groups may be, for example, amine or thiol functional groups. According to some aspects, the multi-functional PEG-based or poly(ethylenimine)-based polymer may comprise a plurality of 2-20 functional groups, e.g., 4, 6, 8, or 15 functional groups. Exemplary structures representing a branched poly(ethylenimine) having amine functional groups (FIG. 1A) and a branched poly(ethylenimine) having thiol functional groups (FIG. 1B) may be used in the present disclosure. In some aspects, the macromer is dissolved in a buffer. Exemplary buffers in which the macromer may be dissolved include, but are not limited to, borate buffers. For example, the borate buffer may have a pH of approximately 8.5-9.0.

A second component of the system may include a crosslinking agent. Exemplary crosslinking agents include, but are not limited to, PEG-based polymers. For example, the PEG-based polymer used as the crosslinking agent may have a molecular weight greater than 3000 Da, such as about 3200 Da to about 3500 Da, e.g., approximately 3400 Da. According to some aspects of the present disclosure, the crosslinking agent have a molecular weight ranging from about 3000 Da to about 3800 Da, from about 3200 Da to about 3500 Da, or from about 3400 Da to about 3800 Da. In some examples, the crosslinking agent may be a PEG-based polymer that comprises one or more N-hydroxysuccinimide or maleimide functional groups. The N-hydroxysuccinimide or maleimide group may react with a macromer as discussed in further detail below. Exemplary structures representing a N-hydroxysuccinimide-PEG polymer and a maleimide-PEG polymer are shown in FIGS. 3A and 3B, respectively. However, it is noted that suitable crosslinking agents, e.g., PEG-based polymers, are not limited to N-hydroxysuccinimide or maleimide functional groups. PEG-based polymers suitable for the present disclosure may include other functional groups that may react with the macromer of the first component the system for forming a gel.

In some aspects, the crosslinking agent may be provided in solution with a buffer, e.g., the crosslinking agent being dissolved in a buffer. For example, the buffer may be a phosphate buffer, e.g., phosphate-buffered saline (PBS). The buffer in which the crosslinking agent is provided, e.g., dissolved, may have a pH that is lower than the buffer in which the macromer is dissolved. For example, the crosslinking agent may be provided, e.g., dissolved, in a solution of PBS having a pH of approximately 6.0-6.5. Thus, the system for forming a gel according to the present disclosure may be pH-activated and may comprise at least two buffers, one having a higher pH than the other.

The crosslinking agent and the macromer may be present with a functional group ratio (e.g., N-hydroxysuccinimide:amine, N-hydroxysuccinimide:thiol, maleimide:amine, maleimide:thiol, etc.) of approximately a 3:2-7:3 molar ratio, for example a 2:1 molar ratio, respectively. The gel may be an aqueous composition in which a combined content of the crosslinking agent and the macromer is at least 15% by weight, with respect to the total weight of the composition. For example, the content of the crosslinking agent may be between approximately 10-20 wt %, in relation to the total weight of the composition, e.g., ranging from about 10 wt % to about 15 wt %, from about 12 wt % to about 18 wt %, or from about 15 wt % to about 20 wt %. Additionally or alternatively, the content of the macromer may be between approximately 5-10 wt %, in relation to the total weight of the composition, e.g., ranging from about 5 wt % to about 8 wt %, or from about 7 wt % to about 9 wt %. As discussed above, the first and second part solutions may comprise at least two different buffers, e.g., a first buffer suitable for the crosslinking agent, and a second buffer suitable for the macromer. According to some aspects, the first buffer comprises a phosphate buffer and the second buffer comprises a borate buffer. The aqueous composition may include any suitable salts for the buffers. It is noted that the mechanical properties of the gels, e.g., hydrogels, formed by the compositions herein may be at least partially determined by the amounts of macromer and/or crosslinking agent. For example, the gel strength may increase as the content of the macromer and crosslinking agent in the aqueous composition increases. Thus, for example, a composition comprising about 20 wt % or about 25 wt % of combined macromer and crosslinking agent, in relation to the total weight of the aqueous gel system, may form a gel that has a higher gel strength than a gel formed from a composition comprising about 15 wt % of combined macromer and crosslinking agent.

The components of the composition or system (e.g., the macromer, the crosslinking agent, and respective buffers) may be mixed together.

When under physiological pH, the functional groups of the crosslinking agent and the functional groups of the macromer may react with one another via chemical conjugation, thereby allowing for immediate gelation. For example, when the composition is at a pH of about 7 to about 7.5, the macromer and crosslinking agent may react to form a gel. In some aspects, the gel may form within 20 seconds, within 15 seconds, within 10 seconds or within approximately 5 seconds, upon mixing a first component comprising the macromer with a first buffer with a second component comprising the crosslinking agent with a second buffer. For example, the gel may form in a time ranging from about 1 second to about 15 seconds, from about 3 second to about 8 seconds, from about 5 seconds to about 10 seconds, or from about 2 seconds to about 5 seconds. The resulting gel, e.g., hydrogel, may be dissolvable, e.g., passively within the physiological environment over time, or on demand, such as by application of an agent capable of disrupting the hydrogel network. For example, the gel may dissolve within a time period of about 10-30 minutes. Dissolution may be measured within a laboratory environment, for example, by measuring rheology when submerging the gel in an aqueous solution.

Gels formed from the macromer and crosslinking agent may exhibit desired properties. For example, storage moduli of the resulting gel, e.g., hydrogel (as a measure of gel strength), may range from about 2.0-10.5 kPa, such as from about 2.5 kPa to about 10 kPa, from about 5 kPa to about 8 kPa, or from about 3.5 kPa to about 7.5 kPa. Additionally or alternatively, the gels may retain a gel strength (also referred to herein as storage modulus G′) ranging from about 2000-10,000 Pa, in approximately room temperature settings, for a desired duration of time, such as, e.g., up to 30 days or longer. Further, for example, the gel, e.g., hydrogel, may have a shear force between about 0.03-0.90 N/cm², e.g., between about 0.1-0.6 N/cm², such as ranging from about 0.05 N/cm² to about 0.4 N/cm², from about 0.5 N/cm² to about 0.9 N/cm², or from about 0.75 N/cm² to about 0.9 N/cm², when adhered to tissue, such as tissue of a bodily lumen, e.g., colon tissue of the GI tract.

The gel may additionally or alternatively be formulated to withstand a burst pressure (corresponding to the pressure at which the gel when adhered to tissue will rip or fail) of up to approximately 200 mbar of pressure, e.g., 150 mbar of pressure, for example a burst pressure greater than 1 mbar and less than or equal to 200 mbar (1 mbar=100 Pa). It is noted that the burst pressure of the gel may be measured by a catheter and pressure transducer (including, e.g., Millar catheters equipped with pressure sensors), which may be utilized to measure the baseline pressure and the pressure just before bursting. To measure burst pressure, a gel may be formed in situ in an aperture of a tissue sample and exposed to fluid of increasing pressure up to the point the cohesion of the gel and/or adhesion of the gel to the tissue breaks down to allow fluid to pass through the aperture of the tissue. The pressure corresponding to the maximum pressure of the fluid just before the gel fails is the burst pressure.

Burst pressure of a gel applied to tissue of the GI tract, such as colon tissue, may be measured as follows. First, an aperture having approximate width and length dimensions of 1 mm×5 mm is cut within the tissue (the depth of the aperture corresponding to the thickness of the tissue, approximately 5 mm in the case of colon tissue). The tissue sample is secured over the open end of a container such that an area approximately 2 inches in diameter is arranged as an unencumbered window over the container. Saline solution is introduced into the container and allowed to flow through the aperture to calibrate the pressure sensor to a baseline pressure. The gel is then formed in situ to close the aperture. The saline solution is then introduced into the container and the increasing fluid pressure measured until the gel fails to permit the solution to pass through the aperture to exit the container. The maximum pressure just prior to the saline solution breaking through the gel to exit through the aperture is the burst pressure. In some examples herein, the gel may be formulated to withstand a burst pressure of at least 50 mbar, at least 100 mbar, or at least 120 mbar when adhered to colon tissue. For example, the gels herein may be formulated to withstand a burst pressure of up to approximately 150 mbar when adhered to colon tissue, such as a burst pressure ranging from about 50 mbar to about 150 mbar, from about 100 mbar to about 150 mbar, or from about 125 mbar to about 150 mbar. The burst pressure may be measured against colon tissue as described above, using an aperture size of 1 mm×5 mm.

Burst pressure of a gel used as an artery or other vessel occlusion device may be measured by forming the gel in situ to close the vessel, wherein the vessel has an approximate diameter of 4-6 mm. A syringe pump and pressure transducer may be used (see FIG. 56 and Example 11), wherein D₂O may be pumped through the syringe pump at a rate of 1 mL/min until a leak in the gel sample is observed. The peak pressure detected from the pressure transducer is recorded as the burst pressure (units of pressure, 1 mmHg=133.322 Pa).

Light-Activated Gel Systems

The disclosure also includes compositions and systems formulated to form a gel upon activation by light as a stimulus. In some examples, the composition may be formulated to crosslink and form a cohesive gel when exposed to light, e.g., UV light or visible light. Thus, such compositions and systems may be described as being light-activated. Such compositions and systems may comprise, for example, a macromer, a cross-linking agent, a photoinitiator, and a buffer. The buffer may be any suitable buffer at about or slightly beyond physiological pH, depending on the buffer used. For example, phosphate buffers may be in the pH range of 7.0-8.0.

The macromer may be a multi-functional PEG-based polymer that includes at least one functional group. The PEG-based polymer may be linear or branched. The at least one functional group of the macromer may comprise, for example, a thiol group, or an alkene group such as a vinyl group, an allyl group, an acrylate group, or a norbornene group, among other alkene groups. The functional group of the macromer may be selected based on the desired properties of the gel, including, e.g., gelation time. The number of functional groups of the macromer may be between 4-100, e.g., between 10-50, between 25-65, or between 45-85.

In some examples, the crosslinking agent may be a PEG-based polymer that includes at least one functional group. The functional group of the crosslinking agent may be complementary to the functional group of the macromer so as to crosslink the macromer and the crosslinking agent. For example, the at least one functional group of the crosslinking agent may comprise a thiol group or an alkene group, such as a vinyl group, an allyl group, an acrylate group, a norbornene group, or other type of alkene group. The functional group of the crosslinking agent may be selected based on the desired degradation properties of the gel. In some examples herein, the number of functional groups of the crosslinking agent may be between 2-4, e.g., 2, 3, or 4 different functional groups.

In some examples, the macromer comprises a thiol group and the crosslinking agent comprises an alkene group, or vice versa. For example, the crosslinking agent may comprise a PEG-based polymer that comprises a thiol group, and the macromer comprises an alkene group, e.g., an acrylate group. In another example, the macromer may comprise a PEG-based polymer comprising a thiol group, and the crosslinking agent may comprise a PEG-based polymer comprising an alkene group, such as an allyl ether group.

As mentioned above, the composition may comprise a photoinitiator, e.g., to initiate gelation. Thus, for example, the photoinitiator may be a compound that absorbs light of a given wavelength of light. According to aspects of the present disclosure, the photoinitiator may absorb UV light (e.g., wavelength between about 100-390 nm) or visible light (e.g., wavelength between about 390-800 nm). Examples of photoinitiators suitable for the compositions herein that are activated by UV light include, but are not limited to, 2-hydroxy-4′-(2-hydroxyethoxy)-2-methylpropiophenone (Irgacure 2959) and lithium phenyl-2,4,6-trimethylbenzoylphosphinate (LAP). Gelation activated by UV light may take place immediately, e.g., within approximately 5 seconds of UV light exposure. Examples of photoinitiators suitable for the compositions herein that are activated by visible light include, but are not limited to, Eosin Y. Gelation activated by visible light may take place briefly or immediately after visible light exposure, e.g., within approximately 10 seconds of exposure to visible light. In some examples, the photoinitiator absorbs white light, e.g., a wavelength of about 390 nm to about 700 nm. Thus, when the composition is illuminated with UV light or visible light (e.g., white light), depending on the photoinitiator used, the composition may crosslink to form a gel. The intensity of UV light or visible light may range from about 1 mW/cm² to about 150 mW/cm². For example, the UV light (365 nm) intensity may range from about 4 mW/cm² to about 120 mW/cm², and white light intensity may range from about 10 mW/cm² (e.g., at the maximal absorption of the photoinitiator) to about 45 mW/cm², such as 42.9 W/cm².

In some examples, the composition may comprise an additive to facilitate or enhance photopolymerization gelation kinetics. Exemplary additives include, for example small-molecule additives such as a tyrosine derivative, e.g., tyrosine methyl ester or tyrosine ethyl ester. Such compositions may comprise photoinitiators that absorb visible light, e.g., white light.

The aforementioned components of the macromer, the crosslinking agent, and the photoinitiator, may be present at a combined concentration of about 10-25 wt %, in relation to the total weight of the composition. At higher amounts (e.g., about 20-25 wt %), the composition may have a relatively shorter gelation time and result in a gel with relatively higher elasticity. The content of the crosslinking agent may be between 5-10 wt %, in relation to the total weight of the composition. Additionally or alternatively, the content of the macromer may be between 5-15 wt %, in relation to the total weight of the composition. The molar ratio between the functional groups of the macromer and the functional groups of the crosslinking agent may range between 1:1 to 2:1 in the composition. In some examples, the molar ratio is about 1:1. It is noted that the aforementioned moiety ratio of 1:1 may result in an increase in elasticity, i.e., the gel elastic modulus, compared to the aforementioned molar ratio of 2:1. In some examples, the composition or system may include a macromer that comprises two or more functional groups for every crosslinking agent of the composition or system (e.g., a macromer and crosslinking agent in a 1:1 ratio wherein the macromer comprises at least two functional groups; or a macromer and a crosslinking agent in a 1:2 ratio wherein the macromer comprises at least four functional groups). It is further noted that different stoichiometric amounts of a functional moiety, e.g., the number of functional or reactive groups per macromer, may affect both the mechanical properties and swelling ratios of the resulting gel, e.g., hydrogel. For example, an increase in the number of functional groups on a macromer of a light-activated gel system may result in a stiffer (e.g., more viscous) gel with a lower swelling ratio.

The composition may comprise from about 0.1 mM to about 100 mM of the photoinitiator. Higher photoinitiator concentrations, e.g., about 90-100 mM, may provide for relatively faster gelation kinetics as compared to lower concentrations, e.g., about 0.1-1 mM. In cases in which the composition comprises an additive, the composition may comprise up to 10 mM of the additive, such as, e.g., about 0.1 mM to about 10 mM, about 0.1 mM to about 5 mM, about 1 mM to about 5 mM, or about 0.5 mM to about 1 mM.

The resulting light-activated gel, e.g., hydrogel, may exhibit a number of desired properties beneficial for application to tissue before, during, and/or after a medical procedure. For example, the gel, e.g., hydrogel, may exhibit a gel strength (also referred to as storage modulus G′) between 500-2500 Pa, such as ranging from about 500 Pa to about 1500 Pa, from about 1000 Pa to about 2000 Pa, from about 750 Pa to about 1250 Pa, from about 1750 Pa to about 2500 Pa. The gel strength G′ may be dependent on the concentration and macromer and crosslinking agent in the composition and/or ratios of the components relative to each other. The gel, e.g., hydrogel, may exhibit a swelling ratio mf/mi (the fold change in the weight of the gel due to water absorption, that is, mf being the weight of the gel at a particular time point after submerging the gel in the buffer, and mi being the initial weight of the gel before submerging it in the buffer) ranging from about 1.8 to about 1.9 times the initial mass or from about 2.3 to about 2.4 times the initial mass. The resultant gels may also exhibit relatively low levels of cytotoxicity. For example, the gel may exhibit greater than at least 97% viability over a 24 hour exposure to cell lines such as NIH3T3 fibroblasts.

The following examples are intended to illustrate the present disclosure without, however, being limiting in nature. It is understood that the present disclosure encompasses additional embodiments consistent with the foregoing description and following examples. The present disclosure is not limited to the examples further described below and encompasses additional conditions without departing from the scope of the present disclosure.

### EXAMPLES

### Example 1

An exemplary pH-activated composition (gel system) was prepared ex vivo at room temperature according to Table 1 in a humid environment. A first part solution was prepared by combining an amine terminated PEG-based or poly(ethylenimine) macromer with a borate buffer having a pH of 8.5. Separately, a second part solution including a N-hydroxysuccinimide crosslinking agent dissolved in phosphate buffer having a pH of 6.5 was prepared.

TABLE 1

| | First part solution | Second part solution |
|---|---|---|
| Macromer (mg) | 1.4 | N/A |
| Borate buffer (μL) | 100 | N/A |
| Crosslinking agent (mg) | N/A | 36 |
| Phosphate buffer (μL) | N/A | 100 |

The first part solution and second part solution were mixed together to form an aqueous solution that subsequently formed a gel. The compositions were left to complete gelling for one hour before assessing the resulting properties.

Gel strength (storage modulus G′) of the gel was measured at room temperature (about 22-25° C.) using a TA instruments DHR-2 rheometer and assessed using a strain sweep from 1-100%, at a 1 Hz frequency. The linear viscoelastic region was determined as the strain percentage increased, until the curve exhibited a 10% decline in the slope of the gel strength. A frequency sweep was then performed from 1-10 Hz within the linear viscoelastic region at 3% strain. Frequency sweeps were performed at times t=0, 4 hours, 24 hours, 48 hours, 7 days, and 30 days after submerging the gel in 50 mM PBS until the gel dissolved.

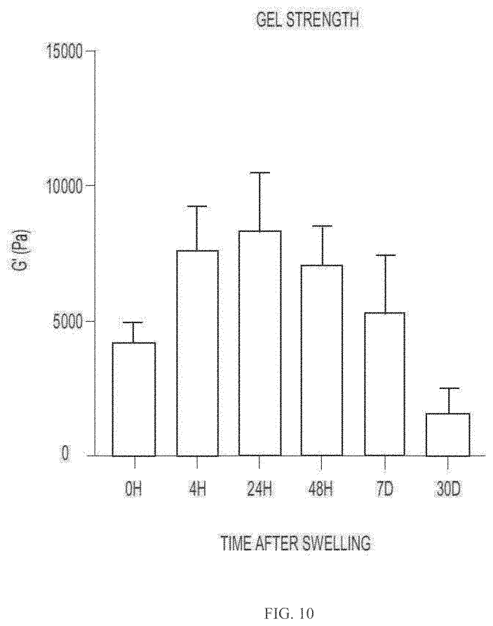

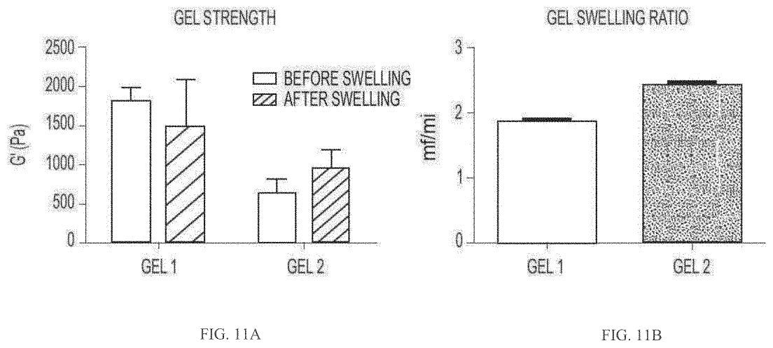

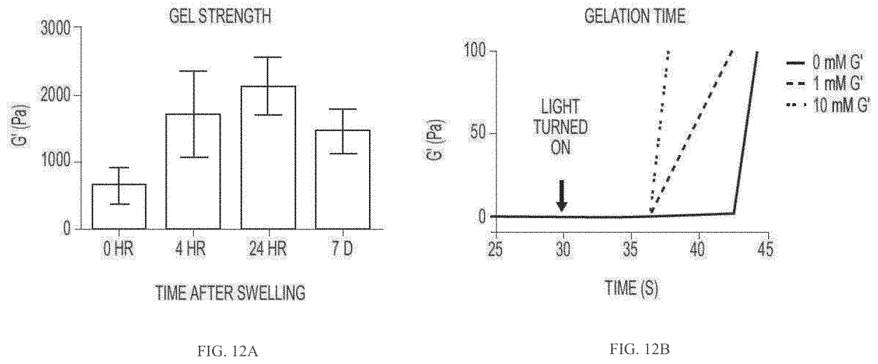

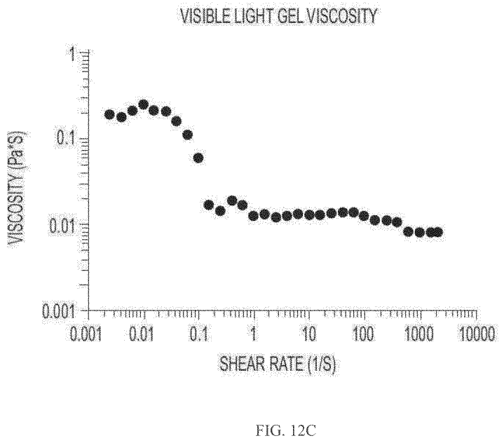

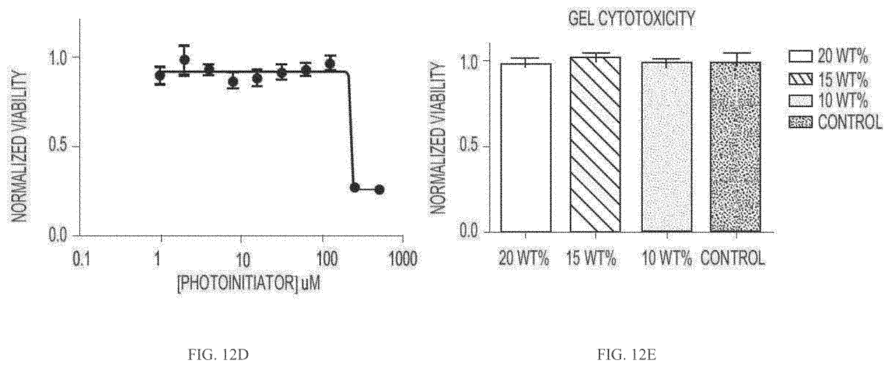

FIG. 10 exhibits the gel strength of the gel during the times noted above. As shown, the gel exhibited gel strength of at least 1,000 Pa for at least 30 days. The gel exhibited an initial gel strength of approximately 4,000 Pa at t=0 and a peak gel strength of approximately 8,000 Pa at t=24 hours. These properties indicate that the pH-activated gel may serve as a lasting protective barrier for tissue for at least a 30-day period.

Gelation time was determined using the inverted tube test, wherein gelation was determined as the time at which the gel no longer runs down the side of the vial when inverted, just after mixing the first and second part solutions. The gelation time was measured at under 1 second.

The swelling ratio was determined as a percentage by weight of the hydrogel after submerging in 50 mM PBS following the equation:

$$Swelling ratio = \frac{m f}{m i} \times 100 \qquad Equation 1$$

with mf being the weight of the gel at that particular time point after submerging the gel in the buffer, and mi being the initial weight of the gel before submerging it in the buffer.

TABLE 2

| | Swelling ratio (%) |
|---|---|
| 15 wt % composition | 220-300 |
| 20 wt % composition | 186-234 |
| 25 wt % composition | 125-162 |

Adhesion measurements were determined by a lap shear test via an Instron® machine, using ex vivo porcine colon tissue. This tissue was dissected into pieces of approximately 2″×1″. The gel was placed between two pieces of colon tissue, and left in a humid chamber for 1 hour to allow for complete gelation. It is noted that in this example, gelation was slowed to about 5-10 minutes to better handle ex-vivo tissue/adhesion measurements. Thus, the tissue sample was left in the chamber for 1 hour to ensure complete gelation. The gel-tissue construct was then mounted on the Instron® and force was continuously measured as the two strips of colon tissue were pulled in opposite directions away from each other at a rate of 10 mm/min until cohesive failure of the hydrogel was observed. Adhesion measurements ranged from 0.03-0.85 N/cm².

### Example 2

Two UV-activated compositions (gel compositions 1 and 2) were prepared ex vivo at room temperature according to Table 3 by combining an alkene containing PEG-based macromer, a thiol containing PEG-based crosslinking agent, and the photoinitiator LAP in PBS having a pH of 7.4. Gel composition 1 was prepared using the PGC-based macromer shown in FIG. 2D, and gel composition 2 was prepared using the PEG-based macromer shown in FIG. 2C. The crosslinking agents were a PEG dithiol crosslinking agent or a 4-arm-PEG-thiol crosslinking agent.

TABLE 3

| | Composition 1 | Composition 2 |
|---|---|---|
| Macromer (mg) | 71.5 | 62.5 |
| Crosslinking agent (mg) | 53.5 | 62.5 |
| Photoinitiator (mg) | 10 | 10 |
| Buffer (mL) | 0.5 | 0.5 |

The compositions were gelled using a handheld 4 W lamp at 365 nm UV light.

Gel strength before and after swelling of the gel in the buffer was measured on a TA instruments DHR-2 rheometer using 8 mm parallel plates at room temperature (about 22-25° C.). A frequency sweep was done with 1% strain and the gel strength G′ identified from the linear viscoelastic portion of the sweep.

FIG. 11A shows the gel strength of the gels before and after swelling. The gel resulting from composition 1 exhibited a gel strength of approximately 1,800 Pa before swelling, and a gel strength of approximately 1,500 Pa after swelling. The gel resulting from composition 2 exhibited a gel strength of approximately 700 Pa before swelling, and a gel strength of approximately 1,000 Pa after swelling. It is noted that composition 1 included a macromer with more reactive moieties per molecule compared to composition 2, which has a macromer with 4 reactive moieties per molecule. Therefore, composition 1 exhibited higher crosslinking density than composition 2, which resulted in stronger gel strength.

Swelling ratio was determined by taking the mass of the initial gel (mi) and then the mass of the gel after swelling in buffer for 24 hours (mf), after blotting off excess buffer according to Equation 1 above.

FIG. 11B shows the gel swelling ratio (expressed as the ratio mf divided by mi in FIG. 11B rather than a percentage) of both gels. The gel resulting from composition 1 exhibited a ratio of approximately 1.9, while the gel resulting from composition 2 exhibited a ratio of approximately 2.4. The difference in swelling ratio between the two compositions is believed also to be a result of the difference in the macromer used, resulting in different crosslinking densities. It is noted that a higher crosslinking density resulted in lower swelling ratio.

### Example 3

An exemplary visible light-activated gel system (composition 3) was prepared ex vivo at room temperature according to Table 4 by combining an alkene containing PEG-based macromer with a thiol containing PEG-based crosslinking agent, the photoinitiator Eosin Y, additive tyrosine ethyl ester, and a phosphate buffer having a pH of 7.0.

TABLE 4

| | Composition 3 |
|---|---|
| Macromer (mg) | 278 |
| Crosslinking agent (mg) | 222 |
| Photoinitiator (mg) | 0.129 |
| Additive (mg) | 0-4 |
| Buffer (mL) | 2 |

The system was gelled using an AmScope 150 W halogen lamp with dual gooseneck fiber-optic illuminators with broad spectrum white light (400-700 nm).

The gel strength was measured at room temperature (about 22-25° C.) as in Example 2 at times t=0, 4 hours, 24 hours, 48 hours, and 7 days, as shown in FIG. 12A.

The gel exhibited an initial gel strength of approximately 600 Pa at t=0 and an increased gel strength over the next 7 days, with a peak gel strength of approximately 2,100 Pa at t=24 hours. The gel exhibited a gel strength of at least 600 Pa for at least 7 days. Thus, these results indicate that the gel may serve as a lasting protective barrier for tissue for at least a 7-day period.

Gelation time was measured on the DHR-2 rheometer at room temperature (about 22-25° C.) with 20 mm parallel plates with the two goosenecks of the halogen lamp directed at the solution before light exposure between the two parallel plates. A time sweep was performed with 1 Hz frequency. The lamp was turned on at 30 seconds, and the time at which gelation occurred thereafter was recorded. Gelation time is shown in FIG. 12B, and as shown, with an increasing concentration of tyrosine ethyl ester (from 0 to 10 mM), there is an increase in the gelation kinetics.

Gel precursor viscosity was determined from a flow sweep on the DHR-2 rheometer at 37° C. using a 50 mm 1.008° cone plate. Results shown in FIG. 12C demonstrate that before photoinitiation, the gel precursor solution demonstrated Newtonian properties and sufficiently low viscosity in the shear rate range above 1/s that allowed the solution to be applied through a long catheter. Further, the solution did not demonstrate significant shear thinning or thickening.

In vitro cytotoxicity of the photoinitiator and the gel were tested using NIH 3T3 fibroblasts cultured in Dulbecco's Modified Eagle's Medium (DMEM) containing 10% fetal bovine serum and 1% penicillin-streptomycin. Cells were seeded in either 96 well plates or 12 well plates at a density of 2,500 cells/well or 25,000 cells/well, respectively, and allowed to adhere overnight. Photoinitiator solutions and the gel solution were sterile filtered using a 0.22 um filter prior to testing in vitro. The gel solution was then gelled within the biosafety cabinet using the 150 W halogen lamp and co-cultured with the cells utilizing a 3 μm pore size transwell insert. Cells were incubated with treatments for 24 hours and then viability was measured using an MTS (3-(4,5-dimethyltriazol-2-yl)-5-(3-carboxymethoxyphenyl)-2-(4-sulfophenyl)-2H-tratrazolium, inner salt) assay (CellTiter 96® AQᵤₑₒᵤₛ One, Promega). Cell viabilities were normalized to control untreated cells.

FIGS. 12D-12E exhibit the results of the above-discussed cytotoxicity tests. The photoinitiator had an IC₅₀ of around 0.223 mM which is above the concentration used in the gel formulations. By using the photoinitiator below the IC₅₀, cytotoxicity issues were not expected with the gels as seen in FIG. 12E, which demonstrated above 97% cell viability for all three weight percent gel formulations. Furthermore, there was no observable difference between the viability for the three gel formulations, indicating that there was no significant toxicity associated with the other components of the gel as their concentration in the solution was increased.

### Example 4

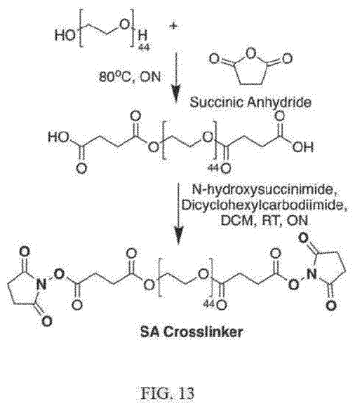

The crosslinking agent shown in FIG. 4A (“SA crosslinker”) was synthesized as illustrated in FIG. 13 as follows.

SA-PEG-SA was first synthesized as follows. Poly(ethylene glycol)(PEG; average Mn 3000 g/mol; Sigma Aldrich) (5 g, 1.6 mmol) was melted in a tri-neck round bottom flask at 120° C. while stirring. Once melted, the flask was put under vacuum and the temperature was then decreased to 80° C. and allowed to stir for 30 minutes. The flask was purged with nitrogen three times. Succinic anhydride (SA) (99%; Aldrich) (0.75 g, 7.5 mmol) was added to the flask. The reaction was stirred under nitrogen for 18 hours. The contents were then dissolved in minimal anhydrous methylene chloride (DCM; 99%; anhydrous; Sigma Aldrich), and precipitated in diethyl ether. Finally, the product was filtered and dried under vacuum for 1 day (white solid, 99% yield). Proton and carbon nuclear magnetic resonance (¹H-NMR, ¹³C-NMR) spectra were obtained on an Agilent 500 MHz spectrometer in CDCl₃ The NMR spectra for the SA-PEG-SA product were as follows: ¹H NMR (500 MHz), CDCl₃: δ 2.62 (m, 8H), 3.64 (overlap, 288H), 4.24 (m, J=4.6 Hz, 4H); ¹³C NMR (500 MHz), CDCl₃: δ 174.0, 172.1, 70.5, 63.8, 29.3, 28.3 ppm. The SA-PEG-SA intermediate was obtained with a reaction yield of 99%.

Next, SA-PEG-SA (4 g, 1.3 mmol) was added to a dry, round bottom flask and dissolved in 15 mL of dry DCM. N-hydroxysuccinimide (NHS; 99%, Sigma Aldrich) (0.4 g, 3.8 mmol) and dicyclohexylcarbodiimide (DCC; 99%; Sigma Aldrich) (0.8 g, 3.8 mmol) were added and the flask was purged with argon. The mixture was stirred for 18 hours at room temperature. Dicyclohexylurea was filtered, the solution was concentrated, and precipitated in diethyl ether. The resulting product SA crosslinker, a white, water-soluble powder, was collected through filtration and dried on vacuum overnight (white solid, 98% yield). The structure was confirmed via ¹H NMR, ¹³C NMR, DSC, and GPC. NMR spectra for the SA crosslinker measured as described above were as follows: ¹H NMR (500 MHz), CDCl₃: δ 2.70 (t, J=1.0 Hz, 4H), 2.77 (t, J=1.0 Hz, 8H), 2.89 (t, J=1.0 Hz, 4H), 3.57 (overlap, 296H), 4.20 (t, J=1.0 Hz, 4H) ppm. ¹³C NMR (500 MHz), CDCl₃: ≡ 170.9, 168.9, 167.6, 70.7, 64.1, 28.6, 26.2, 25.5 ppm.

The molecular weight and polymeric distribution were determined using gel permeation chromatography (GPC) in tetrahydrofuran (THF) as the mobile phase with flow rate of 1.0 mL/min. For the SA crosslinker M_{w}: 2949 g/mol; PDI: 1.02. GPC analyses were performed on an OptiLab DSP Interferometric Refractometer (Wyatt Technology) fitted with two identical Jordi Gel DVB columns (Jordi Labs, 250 mm×10 mm, 10⁵ Å pore size). For the SA crosslinker GPC: Mₙ: 2893 g/mol. Matrix-assisted laser desorption/ionization (MALDI-TOF) was performed on a Bruker autoflex Speed spectrometer equipped with a SMART-beam II and a flash detector. For the SA crosslinker MALDI-TOF (pos): M_{w}: 3600 m/z. Differential scanning calorimeter (DSC) spectra was taken on Q100 TA instrument calorimeter and used to determine melting point (mp). For the SA crosslinker Mp (DSC): 43.5° C.

### Example 5

The crosslinking agent shown in FIG. 4B (“SVA crosslinker”) (average Mn 3400) was obtained from Laysan Bio, Inc. and stored in a glove box. The NMR spectra measured as described above were as follows: ¹H NMR (500 MHz), CDCl₃: δ 1.69 (tt, J=7.3, 7.4 4H), 1.83 (tt, J=6.1, 7.3, 4H), 2.64 (t, J=7.3, 4H), 2.83 (b, 8H), 3.49 (t J=6.1, 4H), 3.63 (m, 300H) ppm. ¹³C NMR (500 MHz), CDCl₃: δ 169.1, 168.6, 70.4, 30.6, 28.4, 25.5, 21.4 ppm. The following was measured for the SVA crosslinker, measured as described in Example 4: MALDI-TOF (pos): M_{w}: 3700 m/z; GPC: Mₙ: 4635 g/mol; M_{w}: 4812 g/mol; polydispersity index PDI: 1.03; Mp (DSC): 47.6° C.

### Example 6

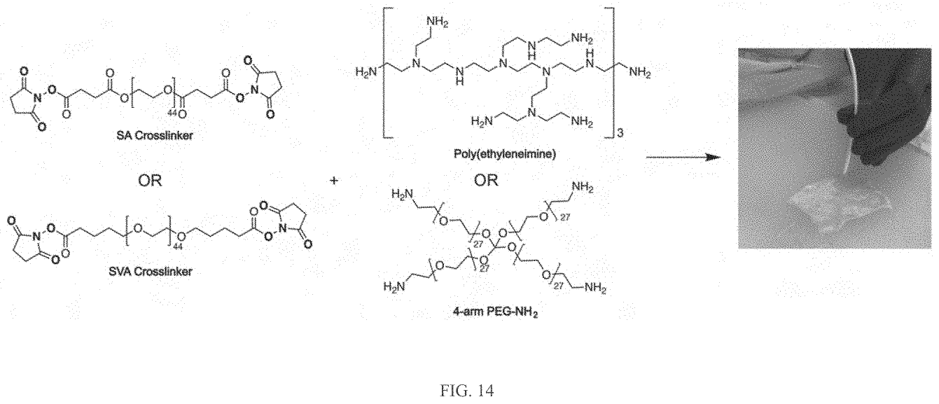

Hydrogels were prepared by combining the crosslinking agents shown in FIG. 4A (“SA crosslinker”) and FIG. 4B (“SVA crosslinker”) with hyperbranched polyethyleneimine (PEI) (average Mn 2000 g/mol; manufacturer Polysciences) or 4-arm PEG-NH₂ HCl salt (4-arm PEG-NH₂) (star polymer; Mn 5000 g/mol; manufacturer JenKem) as illustrated in FIG. 14. Briefly, each PEG crosslinker was dissolved in 0.1 M phosphate buffer, pH 6.5. Each of PEI and the 4-arm PEG-NH₂ was dissolved in 0.3 M borate buffer, pH 8.6. The resulting pH after mixing the crosslinking agent and macromer solutions was adjusted to pH 8.5. The molar ratio of amine to NHS was 1:15 and hydrogels were prepared at 10, 15, or 20 weight percent (wt %). The ratio of amine groups in the macromer to NHS groups in the crosslinking was kept constant, increasing the weight % to increase the amount in solution. Characteristics of the hydrogels were measured and analyzed as discussed in the following sections.

Data was analyzed with Graph Pad Prism 8. For the hydrogel characterization studies, error bars represent standard deviation of the results from three or more replicates. For the bacterial migration studies, error bars represent standard deviation of the results from three biological replicates each performed with three technical replicates or more. A student's T-test was used to compare results and to assess significance. A *p<0.05 is significant.

Gelation Measurements

A relatively fast gelation time (e.g., <3 seconds) may be useful for in situ forming gel, e.g., during polypectomy procedures or other internal wound dressings. For gelation measurements, the crosslinking agent and amine-terminal macromer solutions were mixed and put in a 2 mL glass vial. Gelation was tested using the inverted tube test mechanism. Every 10 seconds the tube was inverted. Gelation was defined by the time at which the solution remained at the bottom of the vial when inverted. All gelation studies were performed at room temperature, 25° C.

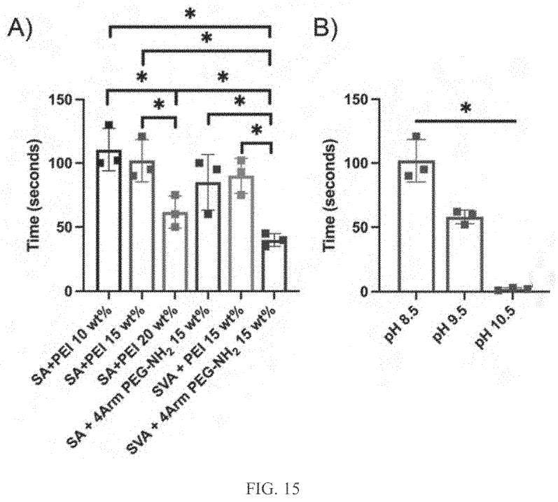

FIG. 15 shows gelation measurements; in panel A) gelation time of hydrogels at varying weight percents and varying formulations, and in panel B) gelation time of SA crosslinker+PEI hydrogels, 15 wt % at increasing pH. *p<0.05. The SA crosslinker+PEI hydrogels gelled faster with increasing weight percent from 10 wt % to 20 wt % as shown in panel A) of FIG. 15 (all hydrogels in panel A) formed at pH 8.5). The increase of gelation time was attributed to a higher concentration of reactive groups therefore favoring a quicker gelation. Next, the gelation times between the SA crosslinker and either the PEI or 4-arm PEG-NH₂ macromer were compared at 15 wt %. The gelation times were similar at approximately 90 seconds (1.5 minutes) suggesting that gelation was independent of the amine macromer.

The SVA crosslinker+PEI hydrogels were found to gel at a similar rate to the SA crosslinker+4-arm PEG-NH₂ and SA crosslinker+PEI hydrogels. However, the SVA crosslinker+4-arm PEG-NH₂ hydrogel was observed to gel faster than the SA crosslinker+4-arm PEG-NH₂. This increase in gelation time was attributed to two factors. The 4-arm PEG-NH₂ macromer contains four, long, amine-terminal arms and has a molecular weight of 5 kDa. The PEI is a more condensed branched macromer with a molecular weight of 2.0 kDa. The long PEG arms of the 4-arm PEG-NH₂ are believed to favor a faster gelation time for the SVA crosslinker+4-arm PEG-NH₂ hydrogel due to increased steric freedom relative to PEI, a branched polymer with shorter arms containing terminal amines and a smaller molecular weight. The steric hindrance observed in the, smaller, branched PEI structure is believed to reduce the ability to readily react with NHS reactive groups in the hydrogel network relative to the higher molecular weight, longer armed, star 4-arm PEG-NH₂ structure.

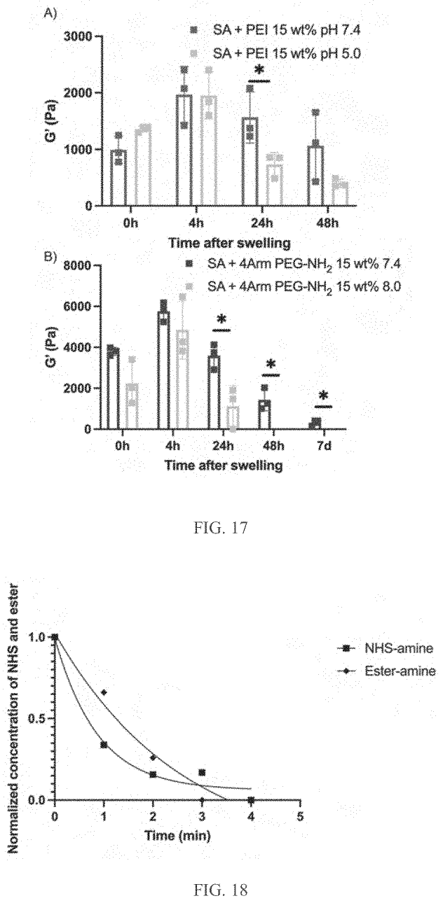

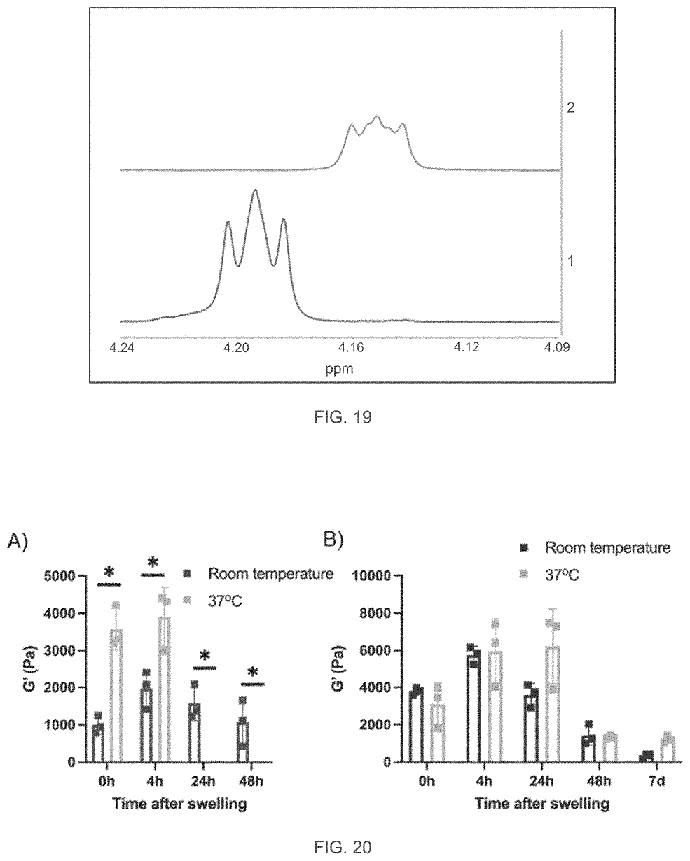

With regards to defects in the hydrogel network, terminal amines favor conjugation at the NHS-ester, however the SA crosslinker contains an internal ester that is also susceptible to macromer amidation and hydrolysis. The preferred site for amidation in the SA crosslinker is at the NHS ester as confirmed via ¹H NMR analysis of a model system, t_{1/2}=0.60 min⁻¹. FIG. 19 shows the change in the NMR spectrum for the reaction, and FIG. 18 shows the change as a function of time, monitoring changes in the signal in the NMR for the NHS ester and the internal ester. While believed to be unlikely, amidation may occur at the internal ester, resulting in defects in the hydrogel network, t_{1/2}=1.8 min⁻¹ (see FIG. 18). Additionally, the half-life for the hydrolysis at the ester linkage is t_{1/2}=<5 minutes at pH 8.0 further providing defects in the hydrogel network. Hydrolysis and amidation of the internal ester are competitive reactions, and therefore may lead to a slower gelation time relative to the SVA crosslinker+4-arm PEG-NH₂ hydrogel.

The effect of pH on gelation time was also evaluated for SA crosslinker+PEI hydrogels at 15 wt %, for pH 8.5, pH 9.5, and pH 10.5, as summarized in panel B) of FIG. 15. The rate of gelation increased with a higher pH of the PEI buffered solution: 100 seconds at pH 8.6, 60 seconds at pH 9.5, and 5 seconds at pH 10.5. For comparison, referring to panel A) of FIG. 15, at pH 8.5, the SA crosslinker+4-arm PEG-NH₂ hydrogel gelled at 80 seconds, the SVA crosslinker+PEI hydrogel gelled at 90 seconds, and the SVA crosslinker+4-arm PEG-NH₂ hydrogel gelled in 45 seconds. Increasing the pH of the PEI solution afforded faster gelation.

Rheological Measurements

Rheological measurements were obtained using a TA instruments DHR-2 Rheometer. Rheological 8 mm parallel plates were used to perform rheological measurements at 22° C. Oscillatory strain sweeps were performed at a frequency of 0.1 Hz from 0.1-10% strain. The linear viscoelastic region was determined from the strain sweep as the strain percent at below which G′ deviates 10° from horizontal. Frequency sweeps were subsequently performed at all time points over 30 days. Strain was set to be within the linear viscoelastic region at 3%, and the frequency ran from 0.1 Hz to 10 Hz following a previously published protocol. Data are expressed as mean±standard deviation (n 3).

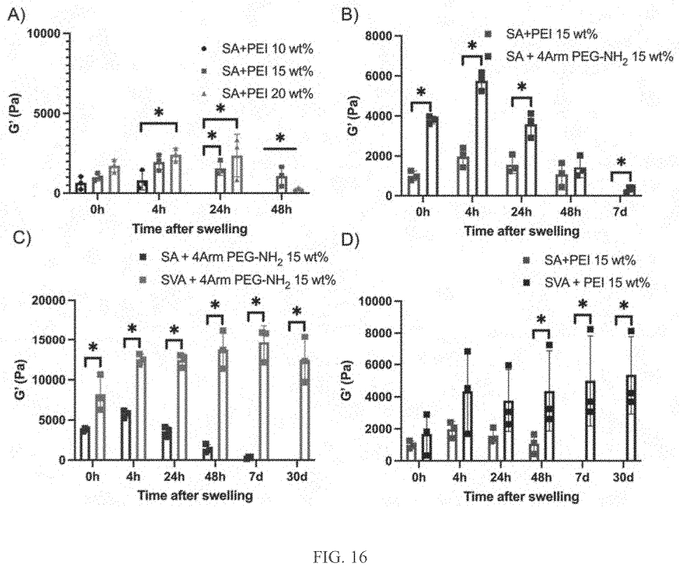

The storage modulus (G′) of the hydrogels was measured at 0 hour, 4 hours, 24 hours, 48 hours, 7 days, and 30 days after swelling in 100 mM PBS, pH 7.4 (applied strain of 3%). The swelling percent and storage modulus (G′) of each hydrogel was measured as an indicator for strength as well as hydrogel swelling over 30 days or until the hydrogel dissolved in 100 uM PBS (pH 7.4). FIG. 16 shows strength (G′) measured for the following hydrogels: panel A) SA crosslinker+PEI at varying weight percents; panel B) SA crosslinker+PEI or SA crosslinker+4-arm PEG-NH₂ at 15 wt %; panel C) SA crosslinker+4-arm PEG-NH₂ and SVA+4-arm PEG-NH₂ at 15 wt %; panel D) SA crosslinker+PEI and SVA crosslinker+PEI at 15 wt %. All rheometry was recorded over time after swelling, *p<0.05.

The SA crosslinker+PEI hydrogels of 10 wt %, 15 wt %, and 20 wt % exhibited average G′ of 638 Pa, 992 Pa, and 2930 Pa, respectively, upon gelation. The hydrogels of 15 wt % and 20 wt % maintained mechanical integrity (G′>300 Pa) through 48 hours, while the 10 wt % SA crosslinker+PEI hydrogels dissolved after 4 hours (G′<300 Pa). To assess the effects of a degradable ester linkage in the crosslinking agent relative to mechanical strength, hydrogels were prepared using the SA or SVA crosslinker and a 4-arm PEG-NH₂ (see panels B) and D)). In comparison, the 15 wt % SA crosslinker+4-arm PEG-NH₂ hydrogels exhibited a G′ of 3814 Pa at time t=0, and maintained integrity through 7 days of swelling with minimal change in G′ through 24 hours and a decrease in mechanical strength at 48 hours and 7 days. The SVA crosslinker+PEI and SVA crosslinker+4-arm PEG-NH₂ hydrogels at 15 wt % exhibited a G′ of 1683 Pa and 7739 Pa, respectively. The G′ of all of the hydrogels initially increased upon swelling (FIG. 16). The SA crosslinker+4-arm PEG-NH₂ hydrogel was found to maintain integrity with the G′ being unchanged (3591 Pa) over 48 hours, and sustained hydrogel morphology over 7 days of swelling, while the SVA crosslinker+4-arm PEG-NH₂ hydrogel maintained mechanical strength over 30 days of swelling (13766 Pa). A similar trend was observed for the hydrogels prepared with PEI, however the G′ remained similar for the SA crosslinker+PEI hydrogel over 48 hours (1380 Pa).

Increased hydrogel weight percent afforded greater G′ and longer sustained mechanical strength as shown for the SA crosslinker+PEI hydrogels (see panel A)). Additionally, the G′ decreased over time for each SA crosslinker+PEI hydrogen, believed to be due to hydrolysis at the internal ester linkage, while G′ remained unchanged for the SVA crosslinker+PEI hydrogels over 30 days of swelling, believed to be due to a lack of degradable linkage within the SVA crosslinker structure (see panel C)).

The increased degradation rate in the SA crosslinker+PEI hydrogels relative to the SA crosslinker+4-arm PEG-NH₂ hydrogels was attributed to a local basic pH within the hydrogel network as a consequence of the PEI. The effect of pH was evaluated by swelling the SA crosslinker+PEI hydrogels in dH₂O, pH 5.0, and the SA crosslinker+4-arm PEG-NH₂ in an aqueous TEA solution (a tertiary amine; comparable [M] to that present in the PEI-based hydrogels) of pH 8.0. FIG. 17 shows, in panel A) storage modulus of SA crosslinker+PEI hydrogels swelled in pH 7.4 and pH 5.0, and in panel B), storage modulus of SA crosslinker+4-arm PEG-NH₂ hydrogels swelled in pH 7.4 and pH 8.0; *p<0.05. The SA crosslinker+PEI hydrogels swelled in pH 5.0 and hydrolyzed in 48 hours, similar to swelling in PBS pH 7.4 (see panel A)). The similar degradation rates were attributed to a strong, basic nature of PEI, and inability to buffer the local pH. By swelling the SA crosslinker+4-arm PEG-NH₂ hydrogels in a solution containing TEA, hydrolysis of the hydrogel accelerated such that the hydrogel degraded in 24 hours compared to 7 days in PBS pH 7.4. This increase in hydrolysis of the SA crosslinker+4-arm PEG-NH₂ hydrogels is consistent with that the local, basic pH of PEI accelerating the hydrolysis of the internal esters in the hydrogel network (see panel B)).

Rheological measurements were performed on the SA crosslinker+PEI hydrogels in dH₂O, pH 5.0, and SA crosslinker+4-arm PEG-NH₂ at pH 8.0. The SA crosslinker+PEI hydrogel exhibited a G′ of 1362 Pa at time t=0 and maintained similar mechanical integrity through 48 hours until significant hydrolysis (loss of hydrogel integrity defined as G′<300 Pa, due to the hydrogel's inability to retain its mechanical structure during rheological measurements when presenting a storage moduli below 300 Pa) regardless of the pH of the water in which the gels were swollen (see FIG. 17). The G′ of the SA crosslinker+4-arm PEG-NH₂ hydrogel formulation was 2239 Pa at time t=0, and it dissolved in aqueous solution pH 8.0 by 48 hours (G′<300 Pa), while the G′ of SA crosslinker+4-arm PEG-NH₂ hydrogels, swelled at pH 7.4, reduced over time but maintained its mechanical integrity through 7 days (G′>300 Pa) (see FIG. 17).

Hydrolysis at the internal ester linkage was confirmed by following a shift in the methylene peak adjacent to the ester from 4.19 ppm to 4.15 ppm on ¹H NMR in a model system. The SA crosslinker was dissolved in a 0.3 M sodium bicarbonate buffered solution in D₂O, pH 8.0. FIG. 19 shows hydrolysis of the internal ester of the SA crosslinker shifted upfield (top) in D₂O, at pH 8.0 relative to unhydrolyzed internal ester of the SA crosslinker (bottom) in unbuffered D₂O based on the change in NMR signal. The half-life (t_{1/2}) was 19.8 minutes for the ester linkage at pH 8.0 in D₂O with TEA present (again, comparable [M] to that present in the PEI-based hydrogels), whereas no ester linkage hydrolysis occurred when TEA was not present (at pH 5 or 6) via ¹H NMR over 24 hours.

Hydrolysis of the SA crosslinker+PEI hydrogel was evaluated at 37° C. to mimic a colon environment. FIG. 20 shows storage modulus G′ values at room temperature as compared to 37° C. for, in panel A) SA crosslinker+PEI hydrogels, and in panel B) SA crosslinker+4-arm PEG-NH₂ hydrogels. The SA crosslinker+PEI hydrogel degraded within 24 hours at 37° C., whereas the SA crosslinker+4-arm PEG-NH₂ hydrogel degraded at the same rate regardless of temperature (room temperature (RT) or 37° C.) and was present for 7 days. The increase in temperature is believed to have further accelerated PEI catalyzed hydrolysis of the hydrogel. The SA crosslinker+PEI hydrogels at 37° C. exhibited greater G′ values (3579 Pa) relative to hydrogels maintained at room temperature, while the SA+4-arm PEG-NH₂ hydrogels were stable over 7 days of swelling regardless of the temperature.

Regardless of the hydrogel composition, an initial increase in storage moduli and swelling occurred after the first 4 hours when the hydrogels were immersed in 100 mM PBS. FIG. 21 reports swelling of hydrogels over 24 hours; SA crosslinker+PEI at 10 wt % hydrogel hydrolyzed at 24 hours, therefore no swelling data was available. Hydrogel swelling percents were reported after 24 h of swelling. The hydrogels swelled until they reached equilibrium at 24 hours or degraded. All the hydrogels swelled to at least 200% of their initial weight in buffer. Hydrogels with increasing weight percent (SA crosslinker+PEI at 10, 15 and 20 wt %) swelled 153%, 259%, and 411%, respectively. The SA crosslinker+4-arm PEG-NH₂ hydrogel swelled 396%. The SVA crosslinker+PEI and SVA crosslinker+4-arm PEG-NH₂ hydrogels swelled 274% and 376%, respectively. It is believed that this absorption characteristic would be useful in covering tissue, such as use as a wound dressing in a polypectomy procedure or other medical procedure.

Adhesion

Adhesion of hydrogels to ex vivo porcine colon tissue was performed on an Instron 5944 Micro-tester. Hydrogels were mixed and placed between two pieces of colon tissue. The colon tissue was divided into 1″×1″ pieces, and the hydrogels gelled directly on the tissue. Upon applying the hydrogel to colon tissue, an additional piece of tissue was placed on top “sandwich style” (tissue-hydrogel-tissue). Tissue adhering to the hydrogel was either the mucosa layer of the colon tissue, or the submucosa layer, obtained by scraping the colon tissue with a scalpel, on each sample. After allowing gelation for one hour in a humid chamber, a lap shear test following ASTM D3165 protocol for adhesion of the hydrogels on colon tissue was performed. Tissue pieces were pulled apart at a rate of 5 mm/min at room temperature until failure in adhesion was detected. Data is expressed as mean±standard deviation (n=3).

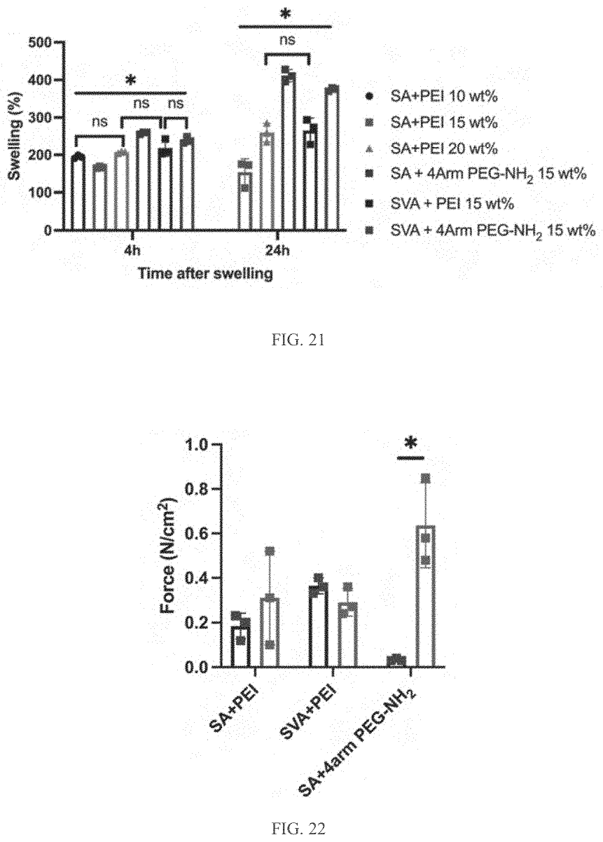

Adhesive strength of the SA crosslinker+PEI, SVA crosslinker+PEI, and SA crosslinker+4-arm PEG-NH₂ hydrogels at 15 wt % was measured on colon tissue with and without the mucosa layer present at 25° C. These hydrogels were chosen to determine whether the presence of the PEI vs 4-arm PEG-NH₂ in the hydrogel alters the adhesion, and if the hydrolyzable SA crosslinker vs non-hydrolyzable SVA crosslinker affects the adhesion. For some samples, the mucosa layer on the colon tissue was removed with a scalpel to expose the submucosa to better model tissue after a polypectomy. The SA crosslinker+PEI, SVA crosslinker+PEI, and SA crosslinker+4-arm PEG-NH₂ hydrogels exhibited mean adhesive strengths of 0.18 N/cm², 0.36 N/cm², and 0.03 N/cm² with the mucosa layer intact, and 0.31 N/cm², 0.29 N/cm², and 0.64 N/cm² without the mucosa layer intact, respectively, as shown in FIG. 22 wherein adhesion of hydrogels with 1 mm thickness on colon tissue with mucosa layer intact (data to the left shown in black) and without mucosa layer (data to the right shown in dark grey). *p<0.05.

The SA crosslinker+PEI and SVA crosslinker+PEI hydrogels adhered the greatest to the tissue with an intact mucosa layer with an adhesivity value of 0.18 N/cm² and 0.36 N/cm², respectively. The SA crosslinker+4-arm PEG-NH₂ hydrogel adhered the strongest to the tissue without the mucosa layer (0.64 N/cm²) (FIG. 22). This difference in adhesion was attributed to hydrogen bonding and charge-charge interactions between the mucosa layer and the cationic PEI compared to the neutral PEG. Mucus is an anionic, hydrophobic, and viscoelastic network with glycoproteins available for hydrogen bonding and electrostatic interactions to molecules such as PEI. PEG, on the other hand, is an uncharged, hydrophilic, and nonfouling; all characteristics understood to retard adhesion to mucus. The SA crosslinker+4-arm PEG-NH₂ hydrogel adhered the strongest to the colon tissue, without the mucosa layer, likely due to the absence of electrostatic interactions with the tissue substrate. It is believed that a force of at least 0.3 N/cm² may be sufficient to maintain adhesion to colon tissue.

Cytotoxicity Studies

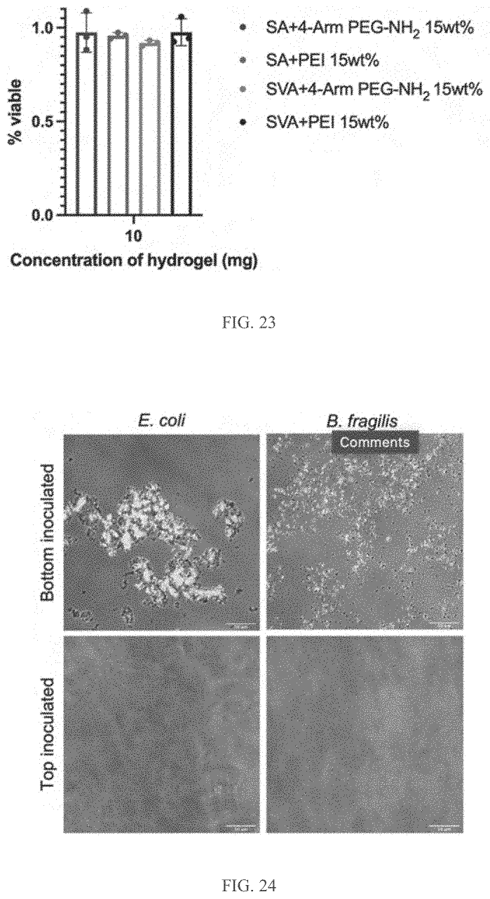

The cell cytotoxicity of hydrogels, at 15 wt % was evaluated against NIH3T3 fibroblasts. Crosslinking agent and PEI solutions were passed through a 0.22 μm PVDF filter prior to mixing and gelation under aseptic conditions. Portions 50 mg, 25 mg, and 10 mg (±2.5 mg) of hydrogel were placed into permeable cell culture inserts (PES, 3 μm pore) (Cell Treat, 230637). Permeable cell culture inserts containing hydrogel samples were incubated in sterile deionized water at 4° C. for 16 hours to allow for swelling. NIH3T3 (ATCC, CRL-1658) were cultured in DMEM+10% BCS+1% PS at 37° C. in 5% CO₂ and 95% humidified air. All cells were passage 4-8 for the experiments. Cells were seeded at 1.25×10⁴ cells/cm² in 24 well plates and allowed to adhere for 16 hours. Media was exchanged and cell culture inserts with swelled hydrogel were transferred into the wells containing adhered cells. Hydrogel samples were briefly equilibrated to 37° C. prior to transfer. Hydrogels were incubated for 24 hours in the presence of cells. Cell culture inserts were removed and a 1:9 dilution of MTS reagent (Promega, G5421) in media was added to each well. Absorbance (490 nm) was measured after 4 hours. Relative cell viability was determined by normalizing absorbance of cells exposed to hydrogel vs a non-exposed control. All experiments were completed in triplicate and error bars represent 1 standard deviation from the mean. All hydrogels were found to be minimally cytotoxic (>88% cell viability) (FIG. 23).

Bacterial Migration

Bacterial migration studies were conducted with isolates of Escherichia coli and Bacteroides fragilis because these microbes are both commonly found in the intestine and are known to cause infections. E. coli is highly mobile and considered to have potential to cross the hydrogel. B. fragilis isolates are known to display multi-drug resistance and cause sepsis. These two common intestinal microbes with pathogenic potential were assessed for the ability to cross the SA crosslinker+PEI and SA crosslinker+4-arm PEG-NH₂ hydrogels.

In vitro testing on agar plates and microscopy studies were performed. An advantage of the agar-based assay is that it detects whether even a few bacterial cells penetrate the hydrogel, because the individual bacteria are allowed to grow for ˜24 hours into a visible colony. Clinical isolates E. coli (ADR129Q-SMC9096) and B. fragilis (CFPLTA004_1B-SMC9107) were obtained from children with cystic fibrosis. Prior to inoculation of hydrogels, E. coli isolates were cultured aerobically overnight in LB (lysogeny broth) and B. fragilis isolates were cultured anaerobically for 48 hours on blood agar (TSA+5% sheep's blood) using the GasPak system. Hydrogel discs (8 mm diameter×2.5 mm height) were placed onto LB agar (for E. coli) or TSA+5% sheep's blood agar (for B. fragilis), and 5 uL of bacteria or PBS was added to the top of each hydrogel. Plates were then incubated at 37° C. for 24 hours aerobically (E. coli) or anaerobically (B. fragilis). After 24 hours, hydrogels were removed, and the agar plates were incubated for an additional 24 hours under the appropriate conditions for each organism to test for bacterial growth below the hydrogel as a measure as to whether the microbes could transit through the hydrogel. After growth, the B. fragilis isolate was scraped into 1 mL PBS and homogenized. 1 mL each of B. fragilis and E. coli were centrifuged for 30 seconds at 16,000×g and resuspended in PBS. Each isolate was then normalized to OD600 of 1.0 in PBS for agar plate experiments and OD600 of 0.1 in minimal medium, as reported (bioproject accession number PRJNA557692), for microscopy experiments. Wells treated with medium-only served to determine background fluorescence, which was subtracted from each sample before analysis.

Microscopy was conducted on a Nikon Eclipse Ti inverted microscope equipped with a Hamamatsu ORCA-Flash 4.0 camera running on Nikon Elements AR. Fast scan mode and 2×2 binning was used and images were acquired through a Plan Fluor 40×DIC M N2 objective. Images were processed in ImageJ in which background was subtracted and signal strength quantified by measuring mean signal intensity/pixel through the Integrated Density (IntDen) function. For microscopy studies, 300 uL of SA crosslinker+4-arm PEG-NH₂ was inoculated into each well of an 8-well plate (Cellvis, catalog #C8-1.5H-N). To visualize bacteria, Syto9 was added to each culture prior to hydrogel inoculation. Bacterial cultures were inoculated either on top of the hydrogel, or below hydrogels that had first been perforated with a pipette tip. Plates were imaged both before and after incubation to determine whether top-inoculated bacteria were able to cross the hydrogel. Results show that these microbes do not transverse across the SA+4-arm PEG-NH₂ hydrogel, indicating its potential for preventing sepsis in vivo. FIG. 24 shows bacterial mitigation by SA crosslinker+4-arm PEG-NH₂ hydrogel. The presence of E. coli (left) and B. fragilis (right) was assessed in perforated hydrogels where bacteria were inoculated into the bottom of the well (top two panels) and non-perforated hydrogels in which bacteria where placed onto the surface (bottom two panels). Three independent experiments were performed, each with three technical replicates. Representative images of merged brightfield and Syto9 staining are shown. Hydrogels were approximately 1 mm in thickness. The E. coli and B. fragilis that were added to the bottom of perforated hydrogels microscopically were observed (FIG. 24, top left and top right, respectively) but absent when added to the top of non-perforated hydrogels (FIG. 24, bottom panels).

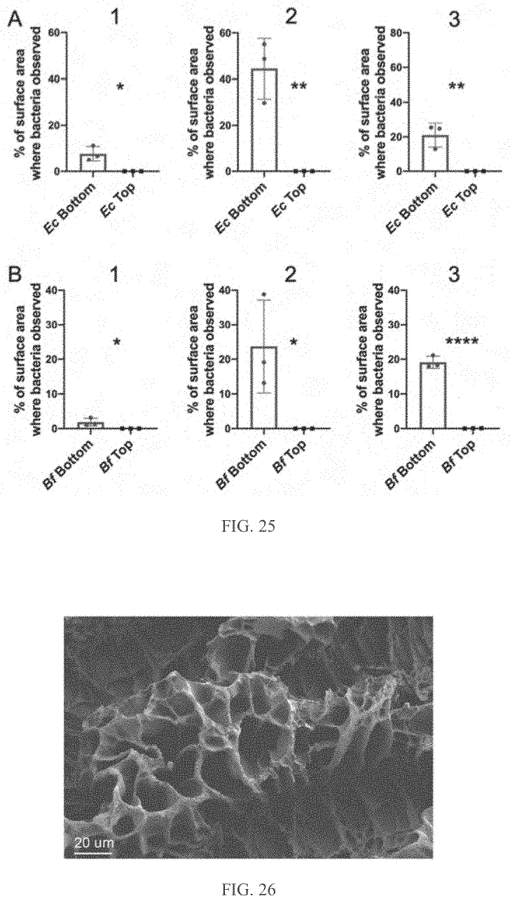

To quantify the impact on bacterial mitigation by the hydrogels, the Syto9 signal intensity was assessed at the bottom of the hydrogels after subtracting background fluorescence from a media-only control. FIG. 25 reports measured surface area of Syto9-stained bacteria 24 hours after the inoculation of E. coli (top graphs A) and B. fragilis (bottom graphs B) on SA crosslinker+4-arm PEG-NH₂ hydrogels. The presence of bacteria was measured in three independent experiments. The surface area occupied by bacteria was compared between perforated hydrogels where bacteria were inoculated into the bottom of the well and non-perforated hydrogels in which bacteria where inoculated onto the surface. Error bars represent standard deviation, *,** and **** indicate a difference of bacterial surface area that is significant at a P value of less than 0.05, 0.01 and 0.0001, respectively. Consistent with imaging in FIG. 24, bacteria inoculated into the bottom of perforated hydrogels show elevated Syto9 staining compared to top inoculated controls (FIG. 25). The SA crosslinker+4-arm PEG-NH₂ hydrogel was found to be an effective barrier to E. coli and B. fragilis for at least 24 hours. In contrast, the SA crosslinker+PEI hydrogels hydrolyzed under laboratory conditions of 37° C., and were therefore unable to be studied.

The lack of bacterial migration through the hydrogel may be a result of hydrogel pore size relative to the bacteria size. The pore sizes of the hydrogels ranged from <1 μm to 20 μm and the pores were not connected giving a mesh-like network as shown by scanning electron microscope for a SA crosslinker+4-arm-PEG-NH₂ hydrogel (FIG. 26). E. coli and B. fragilis are approximately 1.0-4.5 μm in length. Thus the pore size and porosity created a tortuous path for the bacteria to move and inhibited migration. The SA crosslinker+PEI hydrogels hydrolyzed under laboratory conditions of 37° C., and were not suitable for this study.

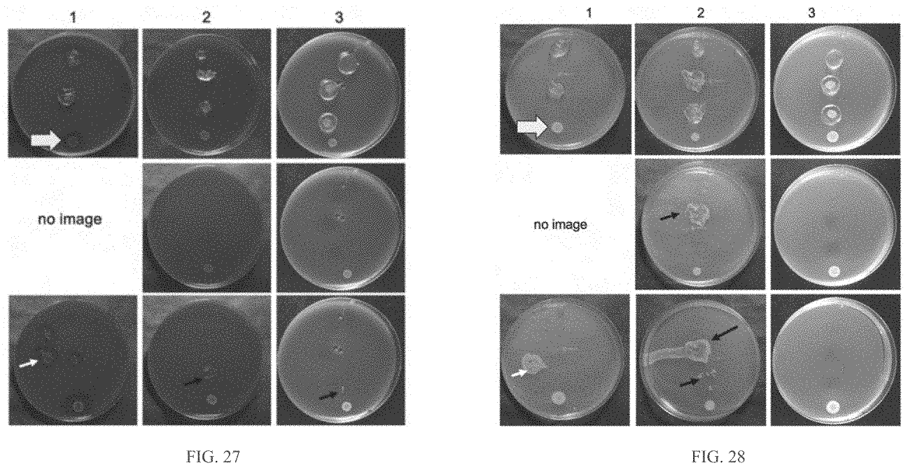

Agar plate assay results are shown in FIGS. 27 and 28. FIG. 27 tests whether B. fragilis can cross SA crosslinker+4-arm PEG-NH₂ hydrogel by placing hydrogels on a TSA+5% sheep's blood agar, applying the bacteria to the surface of the hydrogel and assessing for subsequent B. fragilis growth on the agar after 24 and 48 hours total incubation time. Three independent experiments were performed, with n=3 control and n=4-5 Bacteroides-inoculated hydrogel discs per experiment. For each plate, bacteria was spotted directly onto the plate as a positive control (large arrow, upper left). One representative plate is displayed for each independent replicate. The apical side of each hydrogel was inoculated with either 10 μL sterile PBS or 10 uL B. fragilis culture in PBS at 1 OD₆₀₀/mL. Plates were incubated anaerobically for 24 hours at 37° C. (top row). After 24 hours, hydrogels were removed (middle row), and plates were incubated for an additional 24 hours under the same conditions (bottom row). After 24 hours, B. fragilis growth was apparent on the apical side of the hydrogels but not on the agar, indicating that B. fragilis did not cross the hydrogel in high abundance. After 48 hours, contamination was visible in 11/14 total technical replicates. Of these, 10/11 were most likely edge contamination that occurred while the hydrogel was being removed from the plate (black arrows). In experiment 1, hydrogels were flipped over onto the plate after 24 hours to confirm viability of B. fragilis on the apical side of the hydrogel. Growth derived from the apical side of the hydrogel at 48 hours indicates the B. fragilis was still viable (small white arrow, lower left).

FIG. 28 shows results for E. coli, for SA crosslinker+4-arm PEG-NH₂ hydrogel by placing hydrogels on a LB agar plate, applying the bacteria to the surface of the hydrogel and assessing for subsequent E. coli growth on the agar plate after 24 and 48 hours total incubation time. Three independent experiments were performed, with n=3 control and n=4-5 E. coli-inoculated hydrogel discs per experiment. One representative plate is displayed for each independent replicate. For each plate, bacteria was spotted directly onto the plate as a positive control (large white arrow, upper left). The apical side of each hydrogel was inoculated with either 10 μL sterile PBS or 10 μL E. coli culture in PBS at 1 OD₆₀₀/mL. Plates were incubated aerobically for 24 hours at 37° C. (top row). After 24 hours, hydrogels were removed (middle row), and plates were incubated for an additional 24 hours under the same conditions (bottom row). After 24 hours, E. coli growth was apparent on the apical side of the hydrogels but not on the agar for those E. coli-inoculated hydrogels that remained intact (n=11/15), indicating that E. coli did not cross the hydrogel in high abundance. At 48 hours, plate contamination was visible for n=8/15 discs (black arrows). All of the 24 hour and the majority of the 48 hour contamination occurred during experiment 2. These hydrogels were slightly thinner than in other experiments and some had melted at 24 hours, which is the most likely cause of contamination. In experiment 1, hydrogels were flipped over onto the plate after 24 hours to confirm viability of E. coli on the apical side of the hydrogel. Growth from the apical side at 48 hours indicates that E. coli was still viable (small white arrow, lower left).

The application and handleability of the hydrogels was investigated by administering the crosslinking agent and macromer components through a dual lumen catheter for subsequent hydrogel formation upon exit at a target site on a sample of colon tissue. A dual lumen catheter was used; the catheter is capable of being inserted through an endoscope into the colon in vivo, eliminating the need for a separate device. Air pressure can be applied through the dual lumen catheter to spray the hydrogel precursor components onto a wound to gel in situ. The two-part hydrogel system was delivered on ex vivo colon tissue. All 12 hydrogel formulations were injected through the dual lumen catheter and subsequently gelled and adhered to colon tissue both with and against gravity.

### Example 7

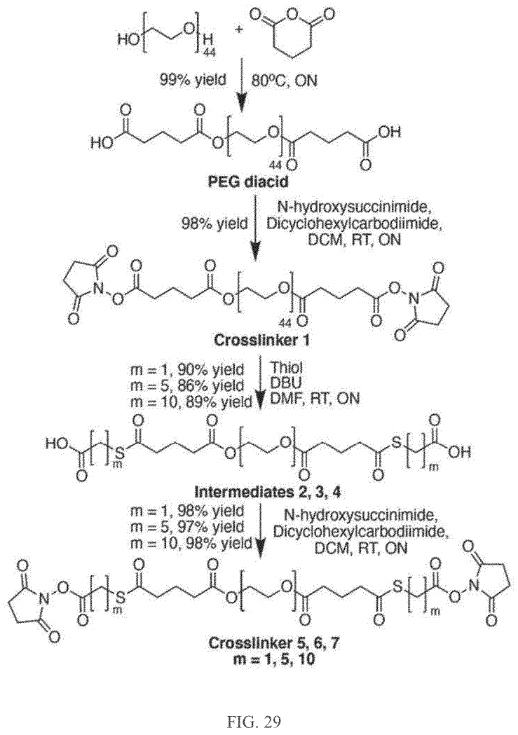

Additional crosslinking agents (crosslinkers 5, 6, and 7) were synthesized starting from PEG (M_{w} 3000) as shown in FIG. 29. Briefly, PEG (M_{w} 3000) was reacted with the appropriate anhydride to form the PEG diacid and subsequently activated with an NHS ester to give crosslinker 1. Crosslinker 1 was reacted with 1,8-diazabicyclo(5.4.0)undec-7-ene (DBU), and the respective thiol-terminal carboxylic acids of 1, 5, and 10 methylenes, to afford intermediates 2, 3, and 4, respectively. Next, the NHS-activated crosslinkers were prepared via dicyclohexylcarbodiimide (DCC) coupling chemistry with NHS and the products purified by precipitation in diethyl ether. The yields were 85-98% for all the reactions. The crosslinking agent structures were confirmed by ¹H NMR, ¹³C NMR, GPC, MALDI and DSC; characterization measurements were conducted as discussed in Example 6. Data was as follows:

PEG Diacid: Synthesis of the PEG diacid compound was based on a previously reported protocol. ¹H NMR (500 MHz), CDCl₃: δ 1.93 (q, J=7.21 Hz, 4H), 2.4 (tt, J=7.21, 8H), 3.62 (m, 292H), 4.22 (tt, J=4.73 Hz, 4H) ppm; ¹³C NMR (500 MHz), CDCl₃: 175.3, 172.8, 70.6, 68.9, 63.4, 33.1, 32.6, 19.9 ppm.

Crosslinker 1. The synthesis of the starting material was based on a previously reported protocol. ¹H NMR (500 MHz), CDCl₃: δ 4.15 (tt, J=3.3, 1.5, 4H), 3.54 (m, 296H), 2.8 (b, 8H), 2.6 (t, J=7.3, 4H), 2.4 (t, J=7.3, 4H), 2.0 (q, J=7.3, 4H) ppm; ¹³C NMR (500 MHz), CDCl₃: 172.3, 169.0, 168.0, 70.5, 69.0, 63.6, 32.4, 29.9, 25.5, 19.7 ppm.

Intermediate 2. Synthesis was based off of a previously reported protocol. ¹H NMR (500 MHz), CDCl₃: δ 4.21 (m, J=4.6, 4.9, 4H), 3.62 (m, 296H), 2.68 (t, J=7.3, 4H), 2.40 (t, J=7.2, 4H), 1.98 (t, J=7.2, 4H) ppm; ¹³C NMR (500 MHz), CDCl₃: 196.8, 172.6, 169.8, 70.6, 69.0, 63.6, 42.3, 32.8, 31.0, 20.5 ppm.

Intermediate 3. In a flame dried flask, 1,8-diazabicyclo(5.4.0)undec-7-ene (265 μL) and 6-mercaptohexanoic acid (122 μL) were added to a solution of crosslinker 1 (1 g) in anhydrous DMF (5 mL). The solution was stirred at room temperature for 16 hours. The organic phase was extracted with a 1M HCl solution, water, and brine. The organic phase was dried over sodium sulfate, filtered, and precipitated in diethyl ether. The precipitate was filtered and dried under vacuum to afford intermediate 3 as a white solid (96% yield). ¹H NMR (500 MHz), CDCl₃: δ 4.22 (t, J=4.8, 4H), 3.63 (m, 308H), 2.86 (t, J=7.2, 4H), 2.61 (t, J=7.3, 4H), 2.38 (t, J=7.4, 4H), 2.30 (t, J=7.4, 4H), 1.97 (t, J=7.3, 4H), 1.60 (m, 8H), 1.39 (m, 4H), ppm; ¹³C NMR (500 MHz), CDCl₃: 198.6, 176.1, 172.7, 70.7, 69.0, 42.8, 33.5, 32.9, 29.2, 28.5, 28.1, 24.2, 20.6 ppm.

Intermediate 4. Synthesis followed the above procedure using 11-mercaptoundecanoic acid (0.190 g) as the thiol source (92% yield). ¹H NMR (500 MHz), CDCl₃: δ 4.22 (t, J=4.9, 4H), 2.85 (t, J=7.4, 7.3, 4H), 2.60 (t, J=7.3, 4H), 2.38 (t, J=7.3, 4H), 2.30 (t, J=7.5, 4H), 1.97 (t, J=7.3, 4H), 1.60 (m, 8H), 1.39 (m, 24H) ppm; ¹³C NMR (500 MHz), CDCl₃: 198.7, 176.5, 172.7, 70.5, 69.0, 63.5, 33.8, 32.9, 29.4, 29.3, 29.2, 29.1, 29.0, 28.95, 28.8, 28.7, 24.7, 20.6 ppm.

Crosslinkers 5, 6 and 7. The synthesis of crosslinkers 5, 6 and 7 was based off of a previously reported protocol (yield 96-98%).

Crosslinker 5. ¹H NMR (500 MHz), CDCl₃: δ 4.16 (t, J=4.3, 4H), 3.92 (s, 4H), 3.57 (m, 257H), 2.78 (b, 8H), 2.67 (t, J=7.3, 4H), 2.34 (t, J=7.3, 4H), 1.95 (q, J=7.3, 4H) ppm; ¹³C NMR (500 MHz), CDCl₃: δ ppm; MALDI-TOF (pos): M_{w}: 3763 m/z; GPC: Mₙ: 5077; M_{w}: 5312; PDI: 1.05; Mp (DSC): 46.06° C.

Crosslinker 6. ¹H NMR (500 MHz), CDCl₃: δ 4.21 (tt, J=1.5, 3.4, 4H), 3.63 (m, 290H), 2.86 (t, J=7.3, 4H), 2.81 (b, 8H), 2.60 (tt, J=2.5, 4.9, 8H), 2.37 (t, J=7.3, 4H), 1.96 (q, J=7.3, 7.4, 4H), 1.74 (q, J=7.4, 7.7, 4H), 1.59 (m, 4H), 1.46 (m, 4H) ppm; ¹³C NMR (500 MHz), CDCl₃: δ 198.6, 172.7, 169.1, 168.4, 70.5, 69.1, 63.6, 42.9, 33.0, 29.1, 28.4, 27.8, 25.6, 24.1, 20.6 ppm; MALDI-TOF (pos): M_{w}: 3807 m/z; GPC: Mₙ: 4999; M_{w}: 5196; PDI: 1.04; Mp (DSC): 45.80° C.

Crosslinker 7. ¹H NMR (500 MHz), CDCl₃: δ 4.22 (m, 4H), 3.62 (m, 278H), 2.85 (m, 8H), 2.70 (t, J=7.2, 7.3, 2H), 2.60 (tt, J=7.3, 4H), 2.45 (t, J=7.2, 7.4, 4H), 2.37 (t, J=7.2, 7.3, 4H), 2.04 (q, J=7.2, 7.4, 4H), 1.95 (m, 4H), 1.71 (m, 2H), 1.52 (m, 4H), 1.25 (m, 10H) ppm; ¹³C NMR (500 MHz), CDCl₃: δ 198.8, 172.7, 169.2, 168.6, 70.5, 69.0, 63.5, 42.8, 32.9, 30.9, 29.5, 29.3, 29.2, 29.0, 28.8, 28.7, 25.6, 24.5, 20.6 ppm; MALDI-TOF (pos): M_{w}: 4210 m/z; GPC: Mₙ: 6038; M_{w}: 6313; PDI: 1.05; Mp (DSC): 47.42° C.

### Example 8

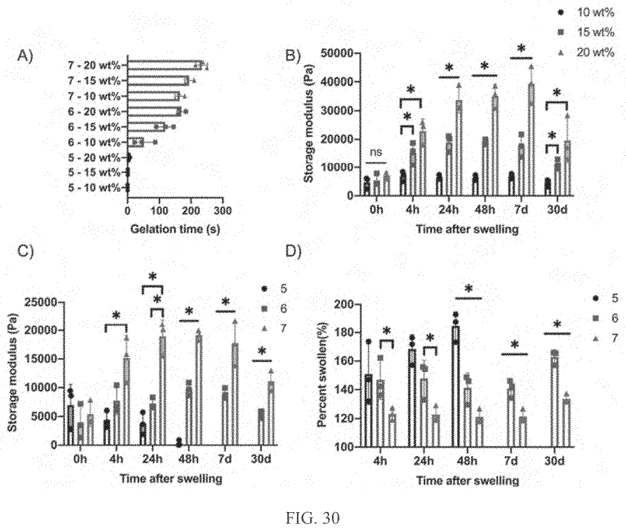

Hydrogels at 10 wt %, 15 wt %, and 20 wt % were prepared by mixing the crosslinking agents of Example 7 (i.e., crosslinkers 5, 6, and 7), dissolved in 0.1 M phosphate buffer pH 6.5, with branched polyethyleneimine (PEI; M_{w} 1800) in 0.3 M borate buffer, pH 8.5. Minimal solubility of crosslinker 7 in buffer was observed and believed to be due to the hydrophobicity of the methylene chains in its structure. In order to overcome the low solubility, crosslinker 7 was dissolved in 0.1 M phosphate buffer pH 6.5 with 50% ethanol prior to mixing it with the PEI solution. The ratio of NHS:NH₂ was 2:1 to ensure amidation of PEI and the respective crosslinking agent. No major difference in hydrogel mechanical properties was observed with a 2:1 or 1:1 NHS:NH₂ ratio. A transparent, solid hydrogel formed within 5 minutes for all compositions (respective hydrogels 5, 6, and 7) as determined by the inverted tube gelation test (see discussion in Example 6). Hydrogel gelation time was found to positively correlate with increasing hydrophobic chain lengths. As shown in FIG. 30, the hydrogels prepared with crosslinkers 5, 6, and 7 gelled in less than 5 seconds, 90 seconds, and 3-5 minutes, respectively. FIG. 30 reports the following: panel A) gelation times of hydrogels at 10 wt %, 15 wt %, and 20 wt %; panel B) hydrogel 7 storage moduli at 10 wt %, 15 wt %, and 20 wt %; panel C) storage moduli of hydrogels 5, 6, and 7, at 15 wt %; panel D) swelling of 15 wt % hydrogels over time. Gelation time was also found to positively correlate with weight percent, meaning the higher the weight percent the longer the gelation time.

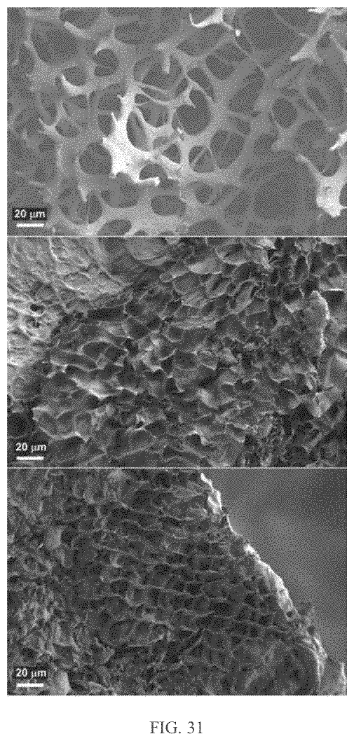

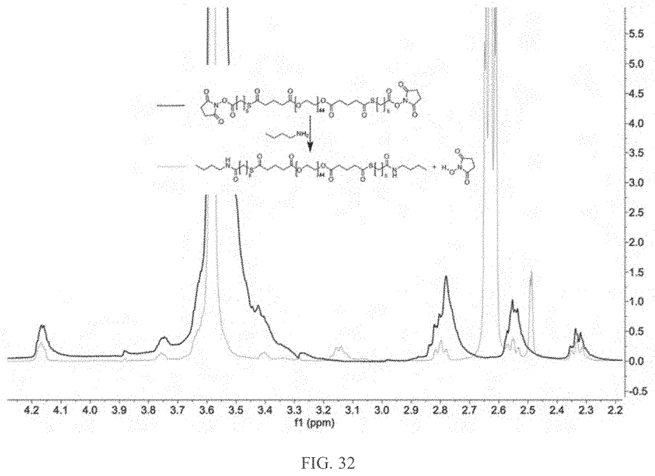

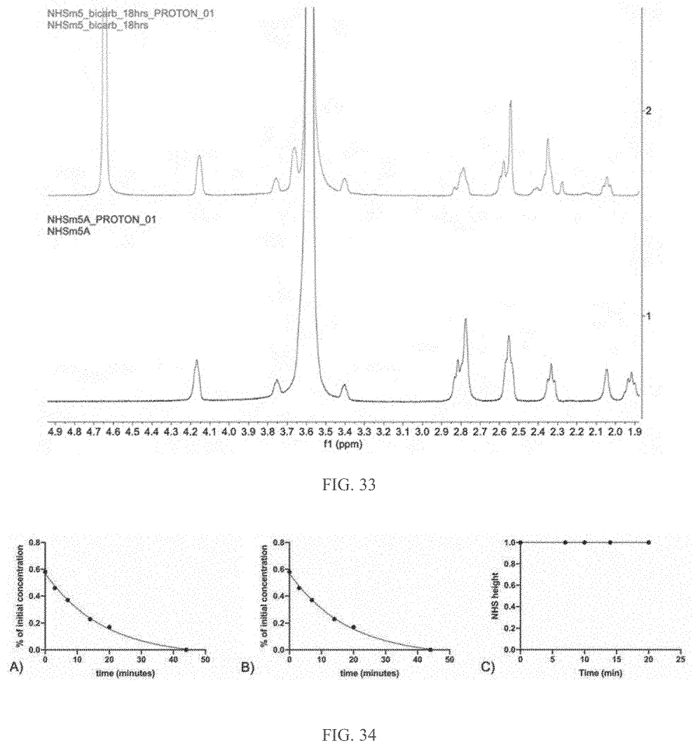

Next, the morphology of the hydrogels was characterized using scanning electron microscopy (SEM). All of the hydrogels possessed pore sizes varying from 5 μm to 100 μm with a honeycomb-like structure. Hydrogel 7, unlike the other hydrogels, exhibited a more lamellar-like structure. FIG. 31 shows SEM images for hydrogels 5 (top), 6 (middle), and 7 (bottom). Because of this observed secondary structure, the critical aggregation concentration (CAC) of crosslinker 7 was assessed using the pyrene assay. A CAC of 0.050 mM was observed, a concentration below that of the hydrogel crosslinker concentration (0.053 mM), indicating formation of a self-assembled structure within the hydrogel itself giving rise to the lamellar structure seen under SEM. From a chemical reactivity perspective, the terminal amines of the PEI may react with the terminal NHS ester or the internal thioesters to form an amide bond. The preferential attack site for the amines was determined via ¹H NMR. Specifically, N-butylamine was used as a model of a primary terminal amine on PEI, and added to an aqueous solution containing crosslinker 6. The amidation reaction was followed via ¹H NMR. Selective reactivity was observed between PEI and the NHS ester on the crosslinkers, and not the internal thiolester (>99% at the NHS site over 20 minutes). Amidation at the NHS ester was confirmed via an upfield shift from the conjugated NHS ester at 2.82 ppm to free NHS at 2.49 ppm on crosslinker 6 while the methylene peak at 2.6 ppm for the thioester does not shift. FIG. 32 shows a representative ¹H NMR spectrum of crosslinker 6 before (bold line) and after (narrow line) reaction with PEI mimetic, N-butylamine. A shift in the NHS peak that is conjugated to crosslinker 6 was observed at 2.78 ppm (bold line) to 2.49 ppm (narrow line) after the NHS ester was cleaved from crosslinker 6 when reacted with N-butylamine. FIG. 33 shows a representative ¹H NMR spectrum of intact crosslinker 6 (bottom) (NHS at 2.78 ppm), and NHS-hydrolyzed (2.54 ppm) crosslinker 6 in 0.3 M sodium bicarbonate buffer, pH 8.0 (top).

The attack of the terminal amine to the NHS-ester occurred quickly, under 10 seconds, however in hydrogels this reaction is likely slower because once one of the amines attacks the NHS-ester, entanglement and solidification is believed to occur with a resulting increase in steric hindrance. Hence the lengthier gelation times. Additionally, a competitive hydrolysis reaction occurred at the NHS ester. FIG. 34 shows rate order of panel A) thioester hydrolysis in crosslinker 5 in 0.3 M Borate buffer, pH 8.0; panel B) thioester hydrolysis in crosslinker 6 in 0.3 M Borate buffer, pH 8.0; and panel C) NHS ester stability in 0.1 M phosphate buffer pH 6.5. Hydrolysis of the NHS ester, however, is negligible at pH 6.5 over 20 minutes, a longer time than sufficient to prepare the hydrogel. (see panel C)). This selectivity of amidation at the NHS ester ensures retention of the internal thioester linkage, allowing for dissolution through cysteine methyl ester (CME).

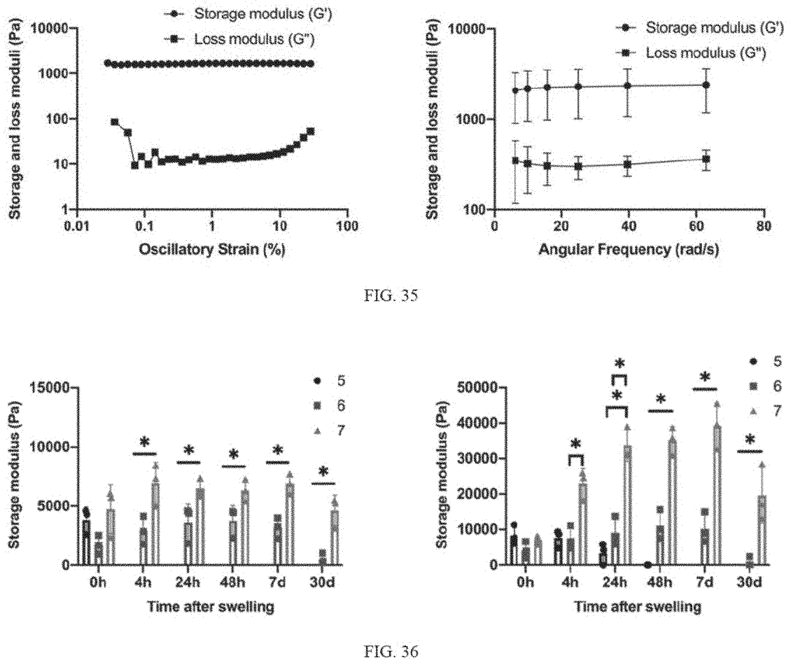

With regards to mechanical properties, strain and frequency sweeps were performed at various time points before and after swelling in 50 mM PBS. First, the linear viscoelastic region was determined using the strain sweep (FIG. 35 (left)). A frequency sweep was performed on all hydrogels with 3% strain from 1 to 10 Hz (FIG. 35 (right)). These hydrogels exhibited viscoelastic, solid-like behavior, storage modulus (G′)>loss modulus (G″).

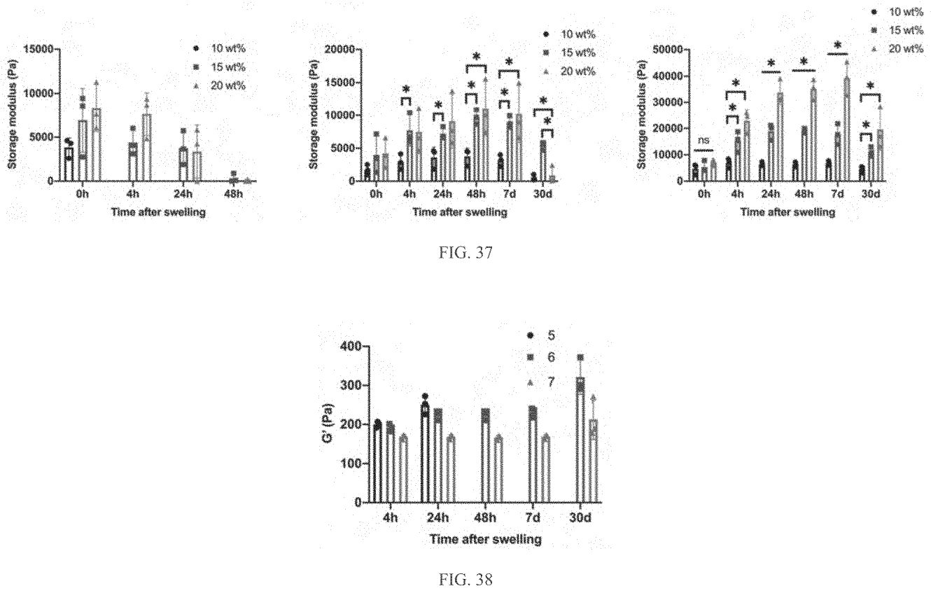

FIG. 36 reports storage modulus of hydrogels 5, 6, and 7 prepared with crosslinkers 5, 6, and 7, respectively at 10 wt % (left) and 20 wt % (right); and FIG. 37 reports storage modulus for hydrogels prepared with crosslinkers 5 (left), 6 (middle), and 7 (right) at 10 wt %, 15 wt %, and 20 wt % over 30 days of swelling or until dissolution. Over 30 days of swelling, the lowest storage modulus was observed for hydrogel 5, sustaining a G′ of below 10 kPa for the duration of time after swelling. The storage modulus of hydrogels prepared with crosslinkers 6 and 7 were each larger, at a peak storage modulus of approximately 12 kPa and 20 kPa, respectively, at 15 wt %. This increase in storage modulus in each hydrogel was attributed to the hydrophobicity of methylenes, such that the longer the methylene chain length, the greater the hydrophobic interactions and a stronger hydrogel. This observation holds true for the weight percent dependence; the higher the weight percent, the greater the storage modulus.

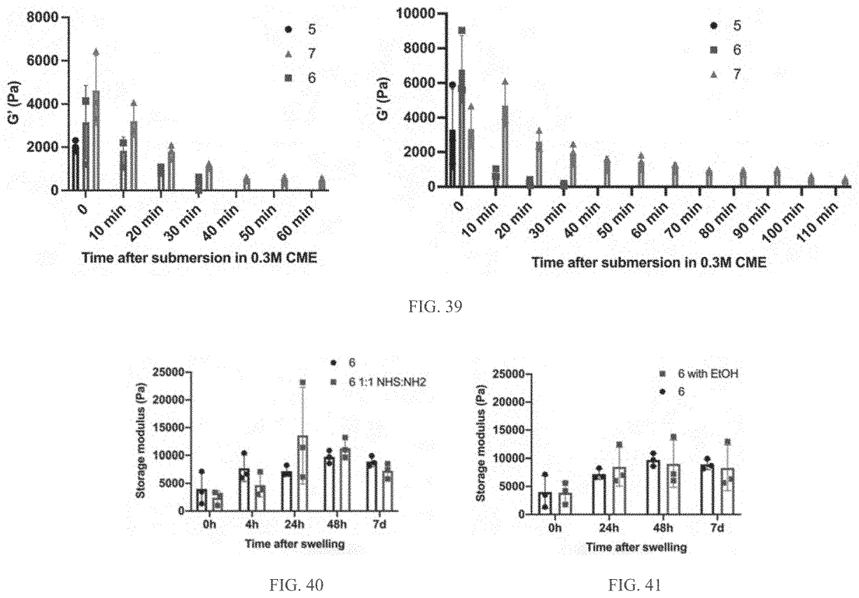

FIG. 38 reports swelling of hydrogels at 20 wt %. FIG. 39 reports dissolution of hydrogels 5, 6, and 7 prepared with crosslinkers 5, 6, and 7 respectively at 10 wt % (left) and 20 wt % (right) upon submersion in 0.3 M CME solution, pH 8.6. FIG. 40 reports rheological measurements on hydrogels prepared from crosslinker 6 with 2:1 (black) or 1:1 (grey) NHS:NH₂ mole ratio. FIG. 41 reports rheological measurements of hydrogels made from crosslinker 6 with and without EtOH.

To ensure that the presence of ethanol did not increase the storage modulus for hydrogels prepared with crosslinker 7, rheological measurements were taken for hydrogels prepared with crosslinker 6 under the same conditions as those hydrogels used for crosslinker 7. No significant difference in storage modulus was observed between hydrogels prepared with or without EtOH, indicating that the buffer conditions did not alter mechanical properties of the hydrogels (FIG. 41).

During the 30 days of swelling, the hydrogels swelled between 150-350% depending on weight percent and hydrophobicity of the hydrogel formulation (FIG. 38). Swelling reached equilibrium after 48 hours for all hydrogels. Hydrogels prepared with crosslinker 7 swelled the least, likely as a consequence of the hydrophobicity within the long methylene chain length, while hydrogels prepared with crosslinker 5 swelled the most.

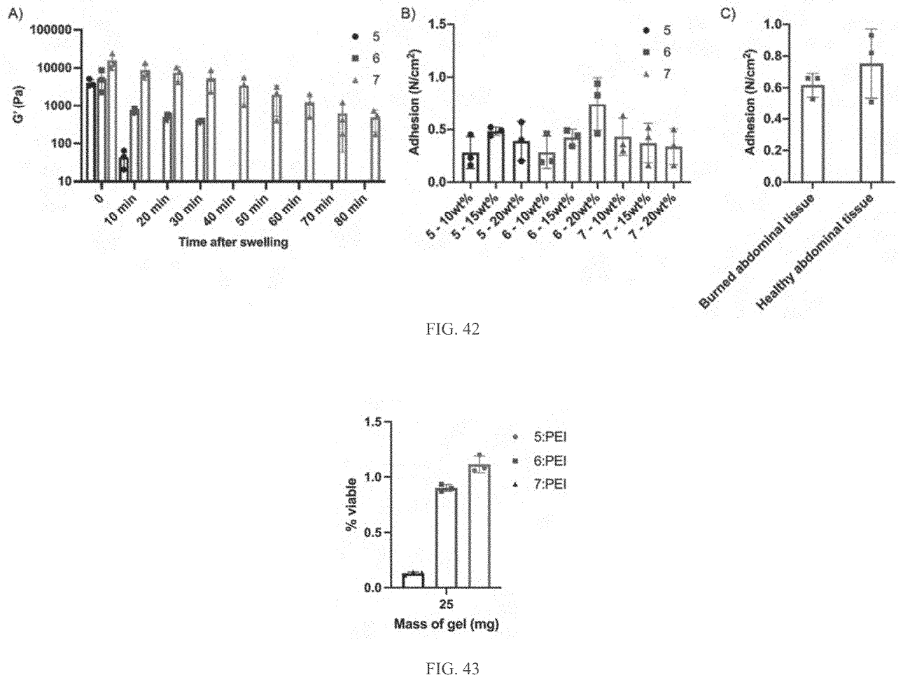

All of the hydrogels underwent hydrolysis over 30 days of swelling as indicated by a loss of gross structure and a reduction in storage modulus over time. Hydrogel 5 exhibited an immediate loss in storage modulus and gross structure while hydrogels 6 and 7 increased in strength as they swelled. However, a reduction in storage modulus was observed in hydrogels 6 and 7 by 30 days post swelling. This loss in structure and mechanical properties was attributed to hydrolysis of the crosslinker. To further characterize the hydrolysis, the rate of crosslinker hydrolysis was measured in 0.1 M sodium bicarbonate buffer, pH 8.0, via ¹H NMR. It was observed that hydrolysis preferentially occurred at the thioester linkage with a rate of k=0.055 min⁻¹ and k=0.003 min⁻¹ for crosslinkers 5 and 6, respectively (FIG. 34) as opposed to the ester linkage between the glutaric acid and PEG on the crosslinker. Hydrogel 7 was stable for over 7 days. The stability of the thioester linkage in crosslinker 7 was attributed to the hydrophobic methylene chain length protecting the adjacent thioester from hydrolysis (see FIG. 9). Aside from hydrolysis, the thioester facilitated hydrogel dissolution through thiol-thioester exchange in the presence of cysteine methyl ester (CME). Upon exposure of the hydrogel to a 0.3 M CME solution at pH 8.6, it is believed that the thiol on the cysteine methyl ester attacked and displaced the internal thioester in the crosslinker. The amine on the internal cysteine methyl ester is believed to have subsequently rearranged to form an amide bond by replacing the thioester (see FIG. 29). This amide bond is believed to prevent re-attack of the original, internal thiol. This dissolution process would fragment the hydrogel network, degrading the hydrogel over time. The storage modulus of the hydrogel in a CME solution was assessed as a function of time at pH 8.6. Complete dissolution, as defined by G′<300 Pa, was found to occur in less than 10 minutes to over 90 minutes depending on the hydrogel formulation and weight percent, with a higher weight percent and longer methylene chain length resulting in an increase in time to complete dissolution. FIG. 42 shows in panel A) dissolution of hydrogels at 15 wt % in 0.3M CME solution; in panel B) adhesion of hydrogels on human breast tissue using a lap shear test; and in panel C) adhesion of 15 wt % hydrogel 6 on burned and unburned human abdominal tissue. See also FIG. 39. Specifically, at 15 wt %, hydrogel 5 dissolved within 10 minutes, while hydrogel 6 dissolved within 30 minutes and hydrogel 7 dissolved within 80 minutes. This trend continued throughout all hydrogels regardless of weight percent. This slower dissolution of hydrogel 7 was attributed to the additional hydrophobic methylenes near the thioester decreasing the local hydrophilicity compared to hydrogels 5 and 6. Due to a competitive reaction at the thioester between hydrolysis of water and thiol-thioester exchange, the rate of dissolution using CME in sodium bicarbonate buffer pH 8.0 was studied via ¹H NMR with crosslinker 6. The decrease in the methylene proton adjacent to the thioester was monitored and the thiol-thioester exchange rate determined to be k=0.084 min⁻¹. This rate was faster than that of hydrolysis and, therefore, interpreted as indicating that thiol-thioester exchange is the preferred mode of dissolution under 0.3 M CME solution conditions.

Adhesive properties of the hydrogels was studied against human skin. A lap shear test was conducted to determine adhesion strength on ex vivo human breast and abdominal tissue. All the hydrogels adhered similarly to tissue with values of approximately 0.5 N/cm² and display cohesive failure at the hydrogel-skin interface (FIG. 42). Additionally, the hydrogel adhered similarly to burned skin as well as healthy skin. The adhesive strength was attributed to physical entanglement between the hydrogel and the human skin.

Prior to the in vivo studies, cytotoxicity was assessed using NIH3T3 fibroblasts. FIG. 43 reports cell viability of hydrogels prepared with crosslinkers 5, 6, and 7 and PEI, against NIH3T3 fibroblasts. Hydrogels 6 and 7 exhibited >85% viability while hydrogel 5 exhibited very low viability, believed to be due to rapid release of glutaric acid and increase in local acidity from the dissolution.

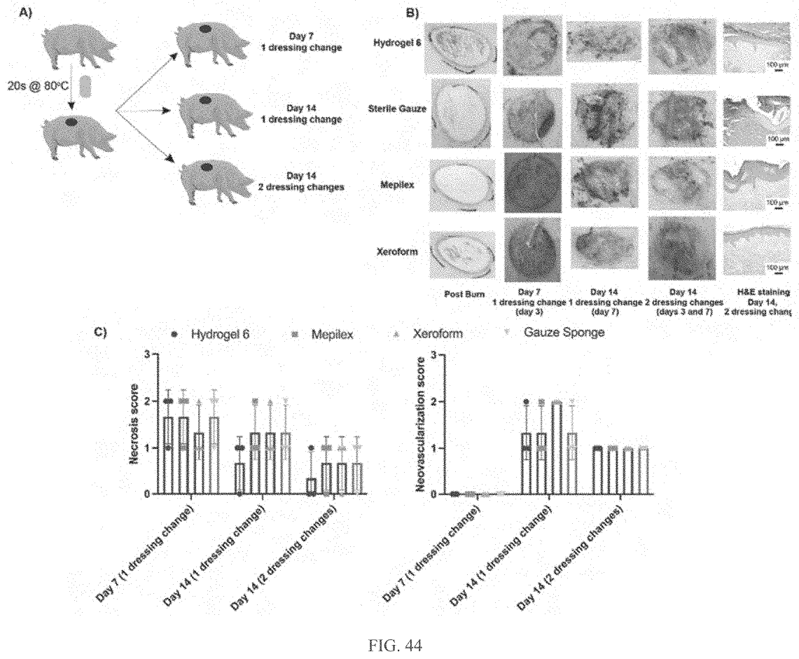

Based on the sum of these results, hydrogel 6 at 15 wt % was selected for in vivo testing. Hydrogel 6 exhibited non-toxicity, storage modulus on the same order as that of human skin, maintenance of mechanical strength and structure over 7 days' time, adhered to skin, swelling, and dissolution in 30 minutes. For the in vivo model, second-degree burns were induced on four pigs by heating a brass cylinder to 80° C. and placing it on the back of the pig for 20 seconds. The treatment groups were assessed at days 7 and 14, with one or two dressing changes as depicted in FIG. 44 to observe any differences in healing between groups. Hydrogel 6 was compared to gauze sponge dressing, Mepilex™, and xeroform. Triple antibiotic ointment was applied to each burn prior to dressing. Post-necropsy, tissue was dissected, and Hematoxalin & Eosin (H & E) staining was performed. FIG. 44 shows, in panel A) experimental schematic; in panel B) representative photographs and histology of burn sites; and in panel C) histology scores of necrosis and neovascularization.

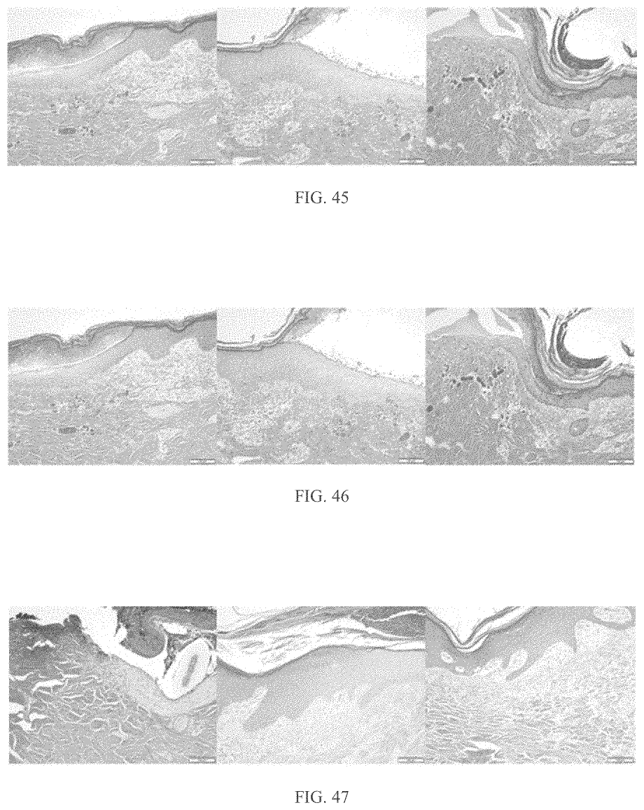

FIGS. 45-49 and Tables 5-9 report data obtained from the samples. FIG. 45 shows H & E of Group 1 for gauze (left), no dressing (middle), and hydrogel dressing (right) (see Table 5); FIG. 46 shows H & E of Group 1 for gauze (left), no dressing (middle), and hydrogel dressing (right) (see Table 6);

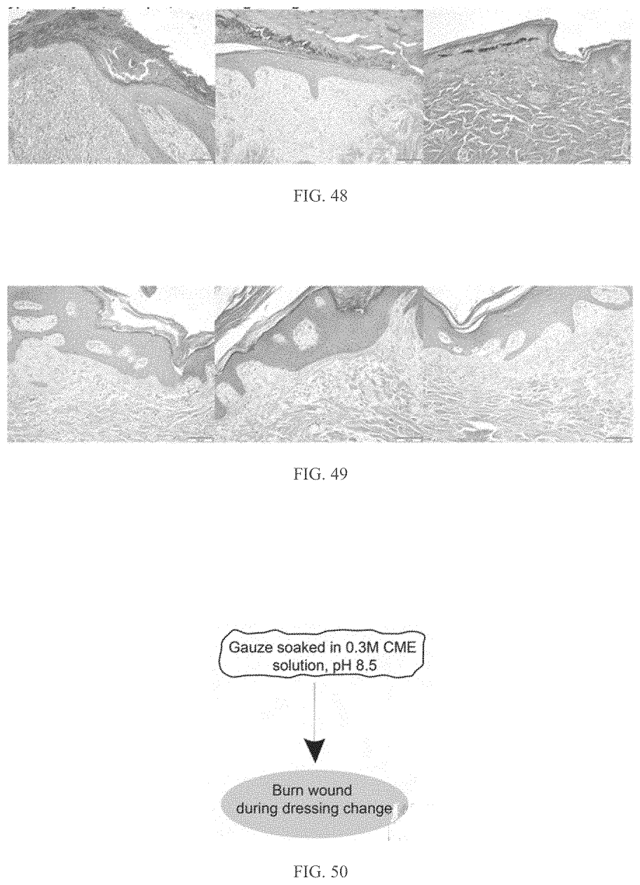

FIG. 47 shows H & E of Group 2 for gauze (left), no dressing (middle), and hydrogel dressing (right) (see Table 7); FIG. 48 shows H & E of Group 4 for gauze (left), no dressing (middle), and hydrogel dressing (right) (see Table 8); FIG. 49 shows H & E of Group 5 for gauze (left), no dressing (middle), and hydrogel dressing (right) (see Table 9). Tables 5-9 show mean±SD, median and incidence of inflammation and inflammatory cell types.

TABLE 5
Day 3, Group 1, no dressing changes.

| | Ionic Hydrogel | | Gauze Sponge | | No Material | |
|---|---|---|---|---|---|---|
| | Dissolving | | (Sterile) | | Used | |
| Parameter | (n = 3) | | (n = 3) | | (n = 3) | |
| Inflammation | 1.00 ± 0.00 | 100% | 1.00 ± 0.00 | 100% | 1.33 ± 0.58 | 100% |
| | 1.00 | | 1.00 | | 1.00 | |
| Neutrophils | 0.33 ± 0.58 | 33% | 0.00 ± 0.00 | 0% | 1.00 ± 1.00 | 67% |
| | 0.00 | | 0.00 | | 1.00 | |
| Histiocytes | 0.00 ± 0.00 | 0% | 0.00 ± 0.00 | 0% | 0.00 ± 0.00 | 0% |
| | 0.00 | | 0.00 | | 0.00 | |
| Lymphocytes | 1.00 ± 0.00 | 100% | 1.00 ± 0.00 | 100% | 1.00 ± 0.00 | 100% |
| | 1.00 | | 1.00 | | 1.00 | |
| Multinucleated | 0.00 ± 0.00 | 0% | 0.00 ± 0.00 | 0% | 0.00 ± 0.00 | 0% |
| Giant Cells | 0.00 | | 0.00 | | 0.00 | |
| Plasma Cells | 0.00 ± 0.00 | 0% | 0.00 ± 0.00 | 0% | 0.00 ± 0.00 | 0% |
| | 0.00 | | 0.00 | | 0.00 | |
| Eosinophils | 1.00 ± 0.00 | 100% | 1.00 ± 0.00 | 100% | 1.00 ± 0.00 | 100% |
| | 1.00 | | 1.00 | | 1.00 | |

TABLE 6
Day 7, Group 3, no dressing changes

| | Ionic Hydrogel | | Gauze Sponge | | No Material | |
|---|---|---|---|---|---|---|
| | Dissolving | | (Sterile) | | Used | |
| Parameter | (n = 3) | | (n = 3) | | (n = 3) | |
| Inflammation | 1.33 ± 0.58 | 100% | 1.33 ± 0.58 | 100% | 1.33 ± 0.58 | 100% |
| | 1.00 | | 1.00 | | 1.00 | |
| Neutrophils | 1.33 ± 0.58 | 100% | 1.33 ± 0.58 | 100% | 1.33 ± 0.58 | 100% |
| | 1.00 | | 1.00 | | 1.00 | |
| Histiocytes | 1.00 ± 0.00 | 100% | 1.00 ± 0.00 | 100% | 1.00 ± 0.00 | 100% |
| | 1.00 | | 1.00 | | 1.00 | |
| Lymphocytes | 1.00 ± 0.00 | 100% | 1.00 ± 0.00 | 100% | 1.00 ± 0.00 | 100% |
| | 1.00 | | 1.00 | | 1.00 | |
| Multinucleated | 0.00 ± 0.00 | 0% | 0.00 ± 0.00 | 0% | 0.00 ± 0.00 | 0% |
| Giant Cells | 0.00 | | 0.00 | | 0.00 | |
| Plasma Cells | 0.00 ± 0.00 | 0% | 0.00 ± 0.00 | 0% | 0.00 ± 0.00 | 0% |
| | 0.00 | | 0.00 | | 0.00 | |
| Eosinophils | 1.00 ± 0.00 | 100% | 1.00 ± 0.00 | 100% | 1.00 ± 0.00 | 100% |
| | 1.00 | | 1.00 | | 1.00 | |

TABLE 7
Day 7, Group 2, 1 dressing changes

| | Ionic Hydrogel | | Gauze Sponge | | No Material | |
|---|---|---|---|---|---|---|
| | Dissolving | | (Sterile) | | Used | |
| Parameter | (n = 3) | | (n = 3) | | (n = 3) | |
| Inflammation | 1.67 ± 0.58 | 100% | 1.67 ± 0.58 | 100% | 2.00 ± 0.00 | 100% |
| | 2.00 | | 2.00 | | 2.00 | |
| Neutrophils | 1.33 ± 0.58 | 100% | 1.33 ± 0.58 | 100% | 2.00 ± 0.00 | 100% |
| | 1.00 | | 1.00 | | 2.00 | |
| Histiocytes | 1.00 ± 0.00 | 100% | 1.00 ± 0.00 | 100% | 1.00 ± 0.00 | 100% |
| | 1.00 | | 1.00 | | 1.00 | |
| Lymphocytes | 1.00 ± 0.00 | 100% | 1.00 ± 0.00 | 100% | 1.00 ± 0.00 | 100% |
| | 1.00 | | 1.00 | | 1.00 | |
| Multinucleated | 0.00 ± 0.00 | 0% | 0.00 ± 0.00 | 0% | 0.00 ± 0.00 | 0% |
| Giant Cells | 0.00 | | 0.00 | | 0.00 | |
| Plasma Cells | 0.00 ± 0.00 | 0% | 0.00 ± 0.00 | 0% | 0.00 ± 0.00 | 0% |
| | 0.00 | | 0.00 | | 0.00 | |
| Eosinophils | 1.00 ± 0.00 | 100% | 1.00 ± 0.00 | 100% | 1.00 ± 0.00 | 100% |
| | 1.00 | | 1.00 | | 1.00 | |

TABLE 8
Day 14, Group 4, 1 dressing change

| | Ionic Hydrogel | | Gauze Sponge | | No Material | |
|---|---|---|---|---|---|---|
| | Dissolving | | (Sterile) | | Used | |
| Parameter | (n = 3) | | (n = 3) | | (n = 3) | |
| Inflammation | 1.33 ± 0.58 | 100% | 2.00 ± 0.00 | 100% | 1.00 ± 0.00 | 100% |
| | 1.00 | | 2.00 | | 1.00 | |
| Neutrophils | 1.33 ± 0.58 | 100% | 2.00 ± 0.00 | 100% | 1.00 ± 0.00 | 100% |
| | 1.00 | | 2.00 | | 1.00 | |
| Histiocytes | 1.00 ± 0.00 | 100% | 1.00 ± 0.00 | 100% | 1.00 ± 0.00 | 100% |
| | 1.00 | | 1.00 | | 1.00 | |
| Lymphocytes | 1.00 ± 0.00 | 100% | 1.00 ± 0.00 | 100% | 1.00 ± 0.00 | 100% |
| | 1.00 | | 1.00 | | 1.00 | |
| Multinucleated | 0.00 ± 0.00 | 0% | 0.00 ± 0.00 | 0% | 0.00 ± 0.00 | 0% |
| Giant Cells | 0.00 | | 0.00 | | 0.00 | |
| Plasma Cells | 0.00 ± 0.00 | 0% | 0.00 ± 0.00 | 0% | 0.00 ± 0.00 | 0% |
| | 0.00 | | 0.00 | | 0.00 | |
| Eosinophils | 1.00 ± 0.00 | 100% | 1.00 ± 0.00 | 100% | 1.00 ± 0.00 | 100% |
| | 1.00 | | 1.00 | | 1.00 | |

TABLE 9
Day 14, Group 4, 2 dressing changes

| | Ionic Hydrogel | | Gauze Sponge | | No Material | |
|---|---|---|---|---|---|---|
| | Dissolving | | (Sterile) | | Used | |
| Parameter | (n = 3) | | (n = 3) | | (n = 3) | |
| Inflammation | 1.33 ± 0.58 | 100% | 2.00 ± 0.00 | 100% | 1.00 ± 0.00 | 100% |
| | 1.00 | | 2.00 | | 1.00 | |
| Neutrophils | 1.33 ± 0.58 | 100% | 2.00 ± 0.00 | 100% | 1.00 ± 0.00 | 100% |
| | 1.00 | | 2.00 | | 1.00 | |
| Histiocytes | 1.00 ± 0.00 | 100% | 1.00 ± 0.00 | 100% | 1.00 ± 0.00 | 100% |
| | 1.00 | | 1.00 | | 1.00 | |
| Lymphocytes | 1.00 ± 0.00 | 100% | 1.00 ± 0.00 | 100% | 1.00 ± 0.00 | 100% |
| | 1.00 | | 1.00 | | 1.00 | |
| Multinucleated | 0.00 ± 0.00 | 0% | 0.00 ± 0.00 | 0% | 0.00 ± 0.00 | 0% |
| Giant Cells | 0.00 | | 0.00 | | 0.00 | |
| Plasma Cells | 0.00 ± 0.00 | 0% | 0.00 ± 0.00 | 0% | 0.00 ± 0.00 | 0% |
| | 0.00 | | 0.00 | | 0.00 | |
| Eosinophils | 1.00 ± 0.00 | 100% | 1.00 ± 0.00 | 100% | 1.00 ± 0.00 | 100% |
| | 1.00 | | 1.00 | | 1.00 | |

Generally, all treatment groups showed mild/moderate necrosis, epidermal ulceration, inflammation, and neovascularization. Hydrogel 6, however, exhibited less necrosis, epidermal ulceration, and inflammation than other treatment groups, with similar neovascularization, and burn depth (mm) and epidermal dermal thickness (mm) to all treatment groups by day 14 (FIG. 44, panel C)). Additionally, all hydrogels showed some re-epithelialization by day 14, with hydrogel 6 exhibiting complete re-epithelialization on two burns, and partial re-epithelialization on one (N=3) after two dressing changes; the only dressing with more than one complete re-epithelialized burn. The only treatment groups with complete re-epithelialization on a burn include hydrogel 6, on day 14, with 1 dressing change, and sterile gauze dressing on day 14, with two dressing changes. While the differences between the groups are not statistically significant, (P>0.05), hydrogel 6 trended towards better performance over conventional gauze, Mepilex™, and xeroform dressings. The spray-on application and removal process of our hydrogels allows ease of application during dressing and debridement removing the need for mechanical debridement and disruption of newly formed tissue. FIG. 50 shows a schematic for dissolution of the hydrogel 6 used as burn wound dressing. Gauze was soaked in 0.3 M CME solution, placed over the hydrogel 6 burn wound dressing for 10 minutes to induce dressing dissolution. Subsequently, the burn wound was wiped with gauze soaked in H₂O and new hydrogel dressing was prepared on top of the wound.

### Example 9

Additional hydrogels according to the present disclosure are prepared. The macromer is either a PEG-based macromer or a poly(1,2-glycerol carbonate) (PGC) based macromer, featuring an alkene functional moiety. The crosslinking agent is a PEG-based crosslinking agent featuring thiol moieties (see Example 9). These components are dissolved in a phosphate buffer solution in the pH range of 7-8 at total polymer concentrations ranging from 10 wt % to 25 wt %. As the weight percentage of the gel solution increases, the gelation time decreases and the gel elastic modulus increases. The molar ratio between alkene and thiol moieties may range between 1:1 to 2:1 in a gel formulation. A ratio of 1:1 results in a small increase in gel elastic modulus compared to a ratio of 2:1. The alkene functional moiety encompasses many different structures, such as, but not limited to, the alkyl ether shown in FIGS. 2C and 2D or the norbornene shown in FIGS. 2E and 2F. The choice of moiety influences gelation time, with norbornene having faster reaction kinetics than the alkyl ether. The macromer may feature between 4-100 alkene moieties per molecule. Macromers featuring higher amounts of alkene moieties result in stiffer gels and lower swelling ratios. The crosslinking agent may contain between 2-4 thiols per molecule. Gel precursor solutions do not solidify until illuminated with either a 365 nm UV light or white light, depending on the photoinitiator used. The photoinitiator is lithium phenyl-2,4,6-trimethylbenzoylphosphinate (LAP) or Eosin Y. The photoinitiator concentrations range from 0.1 mM to 100 mM depending on the photoinitiator and gelation kinetics, with higher photoinitiator concentrations resulting in faster kinetics. Very high concentrations are believed to potentially disrupt the gel macrostructure and risk cytotoxicity. For the visible light system, tyrosine ethyl ester is included up to 10 mM to increase gelation kinetics. These gels form under a wide range of UV (365 nm) intensities from 4 to 120 mW/cm² and white light from 10 mW/cm² (at the maximal absorption of the photoinitiator) to 42.9 W/cm² of full spectrum white light.

For the UV activated hydrogels, the concentrations and ratios of macromer and crosslinking agent can be modified to modulate the storage modulus between 500 Pa and 2,000 Pa. These formulations are single solutions and display a viscosity amenable to application via a single lumen catheter down the length of an endoscope. These formulations utilize stimuli-responsive gelation in response to long wave UV light with fast kinetics, forming a gel within 5 seconds of illumination. The gels adhere to porcine colon tissue and exhibit strong burst pressure when used to seal a small defect. In vivo studies are performed to apply the components using an endoscopic catheter; the resultant gels are still present 2.5 hours after application. The resultant gels have low cytotoxicity, showing greater than 97% viability in NIH 3T3 fibroblasts over 24 hrs. The photoinitiator is used at concentrations below the IC₅₀ in NIH 3T3 fibroblasts and still exhibits fast (<10 seconds) gelation kinetics in response to a broad range of white light sources, such as bike lights, lamps, or endoscopes, when combined with tyrosine ethyl ester up to 10 mM.

### Example 9

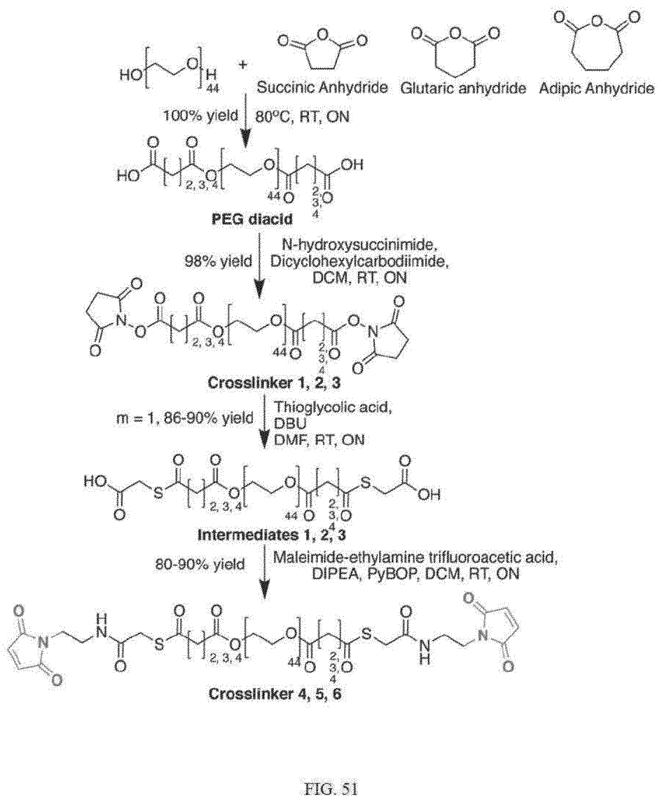

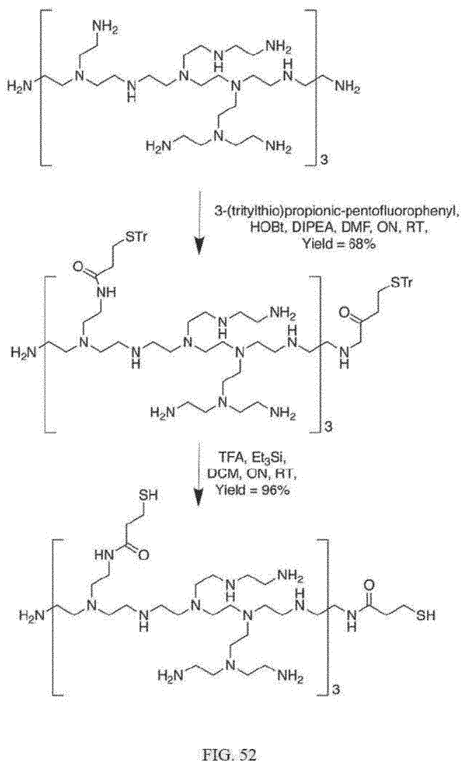

Additional crosslinking agents with thiol moieties were synthesized starting from PEG (M_{w} 3000) as shown in FIG. 51. These crosslinking agents were prepared for studies on formation of hydrogels that form in situ via a Michael addition reaction between a branched PEI-thiol and a bifunctional maleimide-activated, PEG crosslinking agent, discussed further in Examples 10 and 11. Within this crosslinking agent is an internal thioester linkage, susceptible to dissolution via thiol-thioester exchange with a cysteine methyl ester (CME) solution. It is believed that, after thiol-thioester exchange, the primary amine on CME rearranges to form an irreversible amide bond, preventing reformation of the hydrogel after the polymeric network disassembles (see FIG. 8). The PEG crosslinking agents were prepared with methylene chain lengths of 2, 3, or 4, wherein the methylene chain lengths were varied to determine dependence on hydrogel mechanical properties, swelling, dissolution time, and burst pressure. The hydrogels contained an internal thioester for dissolution via thiol-thioester exchange, and maleimide end groups for conjugation with the hyperbranched, poly(ethyleneimine)-thiol (“PEI-SH”).

As summarized in FIG. 51, PEG-diol was reacted with the respective anhydride (succinic anhydride, glutaric anhydride, or adipic anhydride) to obtain the corresponding PEG diacid. Next, the PEG-diacid was functionalized with N-hydroxysuccinimide (NHS) end groups, via DCC coupling to afford crosslinkers 1, 2, and 3. In a flame-dried, roundbottom flask with a magnetic stir bar, crosslinker 1, 2, or 3 (1 g) were dissolved in dimethylformamide (DMF). Thioglycolic acid (68.8 μL) and diisopropylethylamine (DIPEA) (279 μL) were added in that order. Thioglycolic acid was selected because of its hydrophilicity adjacent to the thioester, allowing for fast dissolution times. The reaction was stirred at room temperature, overnight. The organic phase was extracted with a 1N HCl solution, water and then brine. The organic phase was dried with sodium sulfate, filtered through filter paper, and precipitated in diethyl ether to obtain a white powder (98% yield).

Following the previous step, intermediates 1, 2, and 3 were functionalized with maleimide reactive end groups via a peptide coupling method using maleimide trifluoroacetic acid, PyBOP, DIPEA, in dry DCM to obtain the final crosslinking agents, crosslinkers 4, 5, and 6 with methylene chain lengths of 2, 3, and 4, respectively. In a flame-dried, round bottom flask with a magnetic stir bar, intermediate 1, 2, or 3, was dissolved in dry methylene chloride. Maleimide-ethylamine trifluoroacetic acid, DIPEA, HOBt and EDC were added to the reaction. The solution was stirred at room temperature, overnight. The organic phase was extracted using a saturated citric acid solution, water, and brine. The organic phase was then dried with sodium sulfate, filtered through filter paper, and precipitated in diethyl ether to obtain an off-white solid. The solid was dried under vacuum overnight. The solid was then dissolved in water, filtered through a 0.22 μm syringe filter, and lyophilized to obtain an off-white solid (80-90% yield). All yields for the above reactions were above 80%.

Characterization data by ¹H NMR, ¹³C NMR, GPC, and DSC was as follows:

PEG Diacid. This polymer was prepared from a previously published protocol (see also Example 7).

Crosslinkers 1, 2, 3. The synthesis of crosslinkers 1, 2, and 3 were based on a previously published protocol (see also Example 7).

Intermediates 1, 2, 3. Synthesis was conducted as described above. Characterization by ¹H NMR (500 MHz), CDCl₃: Intermediate 1-δ 4.22 (tt, J=4.7 Hz, 4H), 3.62 (m, 310H), 2.93 (t, J=6.8 Hz, 4H), 2.68 (t, J=6.8 Hz, 4H) ppm; Intermediate 2-δ 4.22 (tt, J=4.8 Hz, 4H), 3.63 (m, 308H), 2.86 (t, J=7.2 Hz, 4H), 2.61 (t, J=7.3 Hz, 4H), 2.38 (t, J=7.4 Hz, 4H), 2.30 (t, J=7.4 Hz, 4H), 1.97 (t, J=7.3 Hz, 4H), 1.60 (m, 8H), 1.39 (m, 4H) ppm; Intermediate 3-δ 4.21 (tt, J=4.4, 4.9 Hz, 4H), 3.63 (m, 277H), 2.62 (t, J=6.7, 7.2 Hz, 4H), 2.34 (t, J=6.7, 7.2 Hz, 4H), 1.69 (m, 8H) ppm. Characterization by ¹³C NMR (500 MHz), CDCl₃: Intermediate 1—195.9, 171.5, 70.5, 64.1, 30.9, 29.1 ppm; Intermediate 2-198.6, 172.7, 70.7, 69.0, 33.5, 32.9, 20.6 ppm; Intermediate 3-197.0, 173.0, 70.5, 63.5, 33.7, 31.0, 24.7, 24.0 ppm.

Crosslinkers 4, 5, 6. Synthesis was conducted as described above.

Crosslinker 4. ¹H NMR: δ 6.71 (s, 2H), 6.55 (b, 1H), 4.23 (tt, J=4.2, 4.9 Hz, 4H), 3.62 (m, 322H), 2.96 (t, J=6.8 Hz, 4H), 2.74 (t, J=6.8 Hz, 4H) ppm; ¹³C NMR: 197.5, 171.9, 134.2, 70.5, 64.0, 32.3, 29.1 ppm; M_{w} (GPC, THF): 2868 Da; Mₙ (GPC, THF): 2801 Da; PDI (GPC, THF): 1.02; Melting point (DSC): 41.78° C.; Crystallization point (DSC): 39.9° C.

Crosslinker 5. ¹H NMR: δ 6.72 (s, 2H), 6.51 (b, 1H), 4.23 (tt, J=4.8 Hz, 4H), 3.63 (m, 297H), 2.73 (t, J=7.3 Hz, 4H), 2.42 (t, J=7.2 Hz, 4H), 2.01 (m, J=7.2, 7.3 Hz, 4H) ppm; ¹³C NMR: 198.2, 172.6, 134.2, 70.4, 63.6, 32.8, 32.3, 20.2 ppm; M_{W} (GPC, THF): 3028 Da; Mₙ (GPC, THF): 2955 Da; PDI (GPC, THF): 1.02; Melting point (DSC): 40.22° C.; Crystallization point (DSC): 21.3° C.

Crosslinker 6. ¹H NMR: δ 6.71 (s, 2H), 6.50 (b, 1H), 4.21 (tt, J=, 4H), 2.67 (t, 3H), 2.36 (t, J=, 4H), 1.67 (m, 8H) ppm; ¹³C NMR: 198.6, 173.1, 134.2, 70.5, 63.5, 33.5, 32.4, 24.6, 24.0 ppm; M_{w} (GPC, THF): 3351 Da; Mₙ (GPC, THF): 3162 Da; PDI (GPC, THF): 1.06; Melting point (DSC): 45.04° C.; crystallization point (DSC): 33.5° C.

### Example 10

A thiol-terminated polyethyleneimine (PEI-SH) hyperbranched macromer (FIG. 2G) was synthesized as summarized in FIG. 51 to react with the maleimide terminated PEG crosslinking agents of Example 9. The synthesis of PEI-SH involved reacting a pentafluorophenyl-functionalized, 3-(tritylthio)propionic acid with PEI overnight to obtain a trityl-protected PEI-thiol hyperbranched polymer (“PEI-STr” below) (yield=68%). The trityl groups were then deprotected using TFA and Et₃Si, resulting in the final, PEI-SH hyperbranched polymer (yield=96%). The characterization data, which includes ¹H NMR, ¹³C NMR, GPC, and DSC, is below.

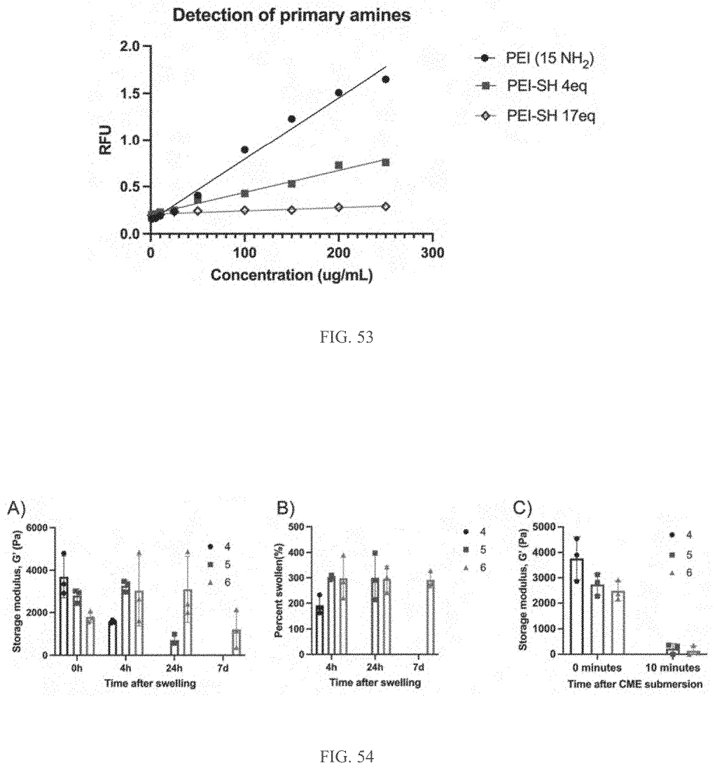

Initially in the PEI-SH synthesis, 15 equivalents of thiol were reacted per PEI molecule to fully thiolate PEI. However, due to the high concentration of thiols per polymer, intra- as well as inter-molecular disulfide bonds formed, as observed visually via a pink solution of PEI-SH in borate buffer, pH 8.6. This minimized the number of available free thiols for a Michael addition reaction with the maleimide-functionalized crosslinking agents of Example 9. The equivalents of thiol reacted with PEI were therefore reduced to minimize the number of inter- and intra-molecular disulfide bonds. The number of free amines was determined via a colorimetric TNBS assay as shown in FIG. 53. The assay was conducted by reacting 0.01% (w/v) solution of 2,4,6-trinitrobenzene sulfonic acid (TNBS) with PEI-SH in 0.1M sodium bicarbonate buffer, pH 8.5. After incubating the solution at 37° C. for 2 hours, the resulting yellow solution was diluted with 10% SDS and 1N HCl to stop the reaction. The absorbance was read at 335 nm, correlating to the number of primary amines present on solution. A standard curve was prepared based on varying concentrations of PEI and fully thiolated PEI-SH where the slope of the RFU vs concentration (ug/mL) graph correlates with the number of free amines on a particular molecule. PEI (MW 1800) has on average 15 free amines, with a TNBS assay slope of 0.007. Fully thiolated PEI-SH, exhibits a slope of 0.000, as expected, signifying zero primary amines on the molecule. The slope of the line representing the PEI-SH prepared with 4 equivalents of tritylthiopropionic acid was assessed. The slope of that line is 0.002, one third of the slope of unfunctionalized PEI. These data confirm thiolating approximately ⅔ of the PEI polymer, meaning 5-6 primary amines remain. This partial functionalization of PEI is expected to minimize intramolecular and intermolecular disulfide bonds and facilitate formation of a hydrogel with the maleimide-functionalized crosslinking agents.

PEI-STr. PEI (3 g) was dissolved in DMF. 3-(tritylthio)propionic-pentofluorophenol (3.4 g), HOBt (3.2 g), and DIPEA (4.7 mL) were added. The reaction was stirred at room temperature, overnight. The reaction was dissolved in methylene chloride, and the organic phase was extracted from sodium bicarbonate, water, and brine. The organic solution was dried over sodium sulfate, filtered through filter paper, and concentrated. The organic solution was precipitated in diethyl ether and dried under vacuum to obtain a light yellow, solid (68% yield). ¹H NMR: δ 8.00 (s, 1H), 7.49-7.10 (m, 48H), 3.65-2.01 (m, 60H) ppm; ¹³C NMR: 162.5, 144.6, 129.5, 127.9, 126.7, 36.5, 35.1, 27.7 ppm.

PEI-SH. In a round bottom flask with a magnetic stir bar, PEI-STr (2 g) was solubilized in a minimal amount of methylene chloride. Trifluoroacetic acid (TFA) (12.3 mL) and triethylsilane (2.7 mL) were added to the stirring solution dropwise, simultaneously. The reaction was stirred for 3 hours at room temperature. Methylene chloride and TFA were removed under vacuum, and redissolved in a minimal amount of methylene chloride. The solution was precipitated in diethyl ether and the product was dried under vacuum overnight. The product was dissolved in 1N HCl, filtered through a 0.22 μm syringe filter, and lyophilized to afford a light-yellow solid (96% yield). ¹H NMR: 7.9 (s, 1H), 3.61-2.49 (m, 217.13H) ppm; ¹³C NMR: 163.1, 162.8, 117.6, 115.3, 39.5, 22.7 ppm; M_{w} (GPC, Aqueous): 5660 Da; Mₙ (GPC, Aqueous): 6994 Da; PDI (GPC, Aqueous): 1.12; Mp (DSC): 15.6° C.

### Example 11

Hydrogels were prepared by combining the crosslinking agents of Example 9 and the macromer of Example 10. The hydrogels were prepared at a ratio of 2:1, crosslinking agent:PEI(SH)₄. The crosslinking agents and PEI-SH were dissolved in 0.1M phosphate buffer pH 6.5 and 0.3M borate buffer pH 8.6, respectively. Each solution was loaded into a dual-lumen syringe with a mixing tip and injected into a cylindrical mold to form a solid hydrogel.

Gelation kinetics were assessed by following the disappearance of the maleimide alkene peak in ¹H NMR upon mixing the maleimide crosslinking agents with mercaptopropionic acid, a PEI-SH mimetic, at 6.70 ppm. An NMR spectrum was recorded every 0.4 s for approximately 20 seconds after injecting 2 equivalents of mercaptopropionic acid, used as a PEI-SH mimetic in situ. No alkene peak was observed at 6.70 ppm immediately following injection of PEI-SH mimetic, exhibiting gelation kinetics faster than 0.4 s.

After gelation, storage modulus of the hydrogel was determined as an assessment of mechanical strength by strain and frequency sweeps to determine the linear viscoelastic region (LVER). The LVER exists to 10 strain %, and is the maximum strain that can be applied to these hydrogels before plastic deformation occurs. A frequency sweep was performed within the LVER at 3% strain, from 0.1-10 Hz. The initial storage moduli of our hydrogels were between 2000-5000 Pa. Upon hydrogel swelling in 50 mM PBS, crosslinkers 4, 5, and 6 exhibited decreasing storage moduli. FIG. 54 shows, in panel A) rheological measurements for hydrogels; panel B) swelling in 50 mM PBS; and panel C) dissolution of hydrogels in 0.3M CME solution. The declining G′ overtime was attributed to degradation of the crosslinking agent via hydrolysis.

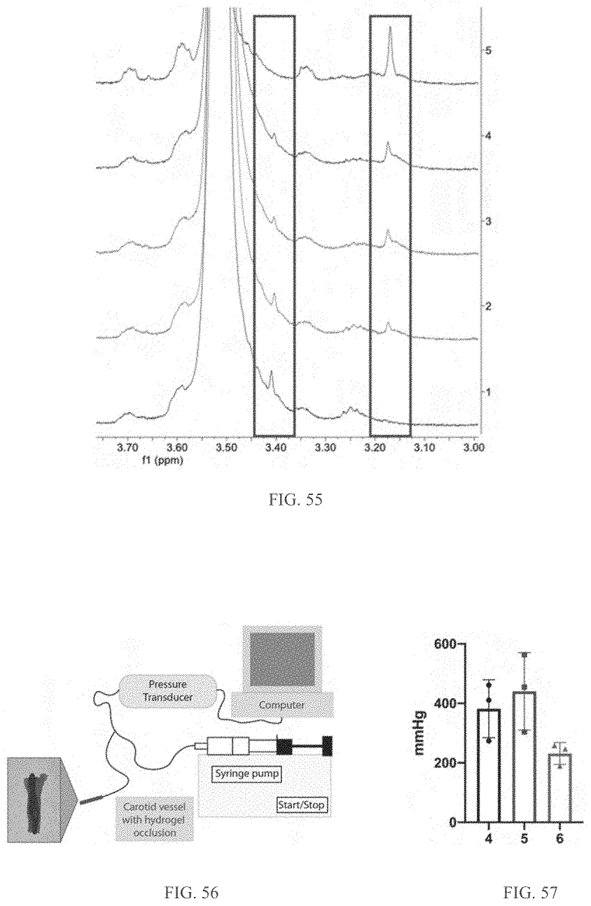

To estimate the rates of degradation and confirm the location of hydrolysis at the internal thioester instead of the ester, the ¹H NMR crosslinking agent spectrum was monitored over 20 minutes in 0.3M sodium bicarbonate buffer, pH 8.0. The methylene adjacent to the internal thiol shifted from 3.41 ppm to 3.17 ppm, while the methylene peak adjacent to the ester linkage at 4.15 ppm, corresponding to the other terminal methylene on the diacid linkage in the crosslinking agent (succinic acid, glutaric acid, adipic acid), did not shift during base-catalyzed hydrolysis. This ¹H NMR shift confirmed selective hydrolysis of the thioester. FIG. 55 shows the ¹H NMR spectra, demonstrating hydrolysis of thioester, observed by shift in methylene adjacent to thiol, from 3.41 ppm (conjugated), to 3.17 ppm (hydrolyzed).

Varying degradation rates, of >4 hours, >24 hours, and >7 days, for hydrogels prepared with crosslinkers 4, 5, and 6, increased relative to the hydrophobic methylene chain lengths of the internal diacid linkage protecting the internal thioesters of the crosslinking agent from hydrolysis. Crosslinker 4 contains an internal succinic acid linkage with two methylenes, while crosslinkers 5 and 6 contain glutaric acid and adipic acid linkages of three and four methylenes, respectively. The longer and more hydrophobic methylene chain length in the crosslinking agent, the more stable the thioester is believed to be against hydrolytic cleavage, resulting in slower degradation rates. The varying degradation rates, relative to the methylene chain length within crosslinkers 4, 5, and 6, allows for tuning the hydrogel mechanical properties through crosslinking agent structure in order to maintain mechanical integrity.

Swelling was observed between 200-400% (FIG. 54, panel B)). Swelling reached its maximum at 24 hours after submersion in 50 mM PBS. Swelling in aqueous solution is believed to be advantageous for a hydrogel due to its ability to expand in size as it absorbs aqueous fluid from the surrounding environment.

On-demand dissolution time of the hydrogels via thiol-thioester exchange was assessed when submerged in a 0.3M cysteine methyl ester (CME) solution (FIG. 8). Frequency sweeps were performed at 10 minute intervals to allow sufficient CME exposure, until the hydrogel network completely disassembled or degraded (G′<300 Pa). Upon thiol-thioester exchange, the hydrogel network disassembled and the amine on the CME rearranged to form an irreversible amide bond, preventing reformation of the hydrogel. Dissolution occurred within 10 minutes or less for all three hydrogel formulations (FIG. 54, panel C)). While the acid linkages (succinic acid, glutaric acid and adipic acid) retard hydrolysis under neutral conditions, the basic conditions of CME solution catalyze the thiol-thioester exchange reaction. Fast dissolution of the hydrogel network via thiol-thioester exchange may be useful in a medical context, e.g., to help to minimize the patient's time under anesthesia.

Prior to ex vivo studies, the cytotoxicity of the hydrogels against NIH3T3 fibroblasts was assessed over 24 hours of exposure. Hydrogels 4 and 5 exhibited a mean cell viability of 60%, while hydrogel 6 showed a mean cell viability of 98%. Low cell viability in hydrogels 4 and 5 was attributed to rapid release of succinic and glutaric acid, due to disassembly of the hydrogel network, and increased local acidity in the confined environment of a trans-well plate.

The hydrogel burst pressure was determined by injecting the macromers into one end of an ex vivo 2 cm porcine carotid artery at a total volume of 1 mL to form the hydrogel. The hydrogel filled the vessels and remained in place. After storing the plugged artery in a humid environment for 30 minutes (e.g., mirroring the time during an exemplary surgical procedure) the vessel was attached to a custom in-house burst pressure system with a pressure transducer connected to a computer, and a syringe pump (FIG. 56). Deionized H₂O was pumped through the vessel at 1 mL/min until a leak was observed and the pressure recorded until failure. The burst pressures for hydrogels 4, 5, 6 prepared with crosslinkers 4, 5, 6 were 382 mmHg, 440 mmHg, and 231 mmHg respectively, up to 4× greater than arterial pressure (120/60) (FIG. 57). A burst pressure of 200-600 mmHg is believed to be sufficient for a hydrogel occlusion device.

It will be apparent to those skilled in the art that various modifications and variations can be made to the present disclosure without departing from the scope of the disclosure. Other embodiments will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of forming a gel, the method comprising:
preparing a composition by combining:
a macromer comprising a first polyethylene glycol (PEG)-based polymer, a poly(ethylenimine)-based polymer, or a poly(1,2-glycerol) carbonate-based polymer, the macromer including at least one first functional moiety;
a crosslinking agent comprising a second PEG-based polymer that includes at least one second functional moiety; and
a photoinitiator; and
activating the photoinitiator via a light source to form the gel, wherein the gel is biocompatible.

2. The method of claim 1, wherein the at least one first functional moiety comprises a thiol group, a vinyl group, an allyl group, an acrylate group, or a norbornene group, and wherein the at least one second functional moiety comprises a thiol group, a vinyl group, an allyl group, an acrylate group, or a norbornene group, the at least one first functional moiety being different from the at least one second functional moiety.

3. The method of claim 2, wherein one of the at least one first functional moiety or the at least one second functional moiety comprises a vinyl group, an allyl group, an acrylate group, or a norbornene group, and the other of the at least one first functional moiety or the at least one second functional moiety comprises a thiol group.

4. The method of claim 1, wherein the macromer, the crosslinking agent, and the photoinitiator represent a total of about 10-25 wt % of the composition, in relation to a total weight of the composition.

5. The method of claim 1, wherein a molar ratio between the at least one first functional moiety and the at least one second functional moiety ranges from 1:1 to 2:1.

6. The method of claim 1, wherein the macromer represents a total of about 5-15 wt % of the composition, in relation to a total weight of the composition; wherein the crosslinking agent represents a total of about 5-10 wt % of the composition, in relation to a total weight of the composition; or wherein the macromer represents a total of about 5-15 wt % of the composition and the crosslinking agent represents a total of about 5-10 wt % of the composition, in relation to a total weight of the composition.

7. The method of claim 1, wherein the gel is formed on tissue of a gastrointestinal tract of a subject during a medical procedure.

8. The method of claim 1, wherein a concentration of the photoinitiator within the composition ranges from about 0.1 mM to about 100 mM.

9. The method of claim 1, wherein the composition further comprises a physiological buffer.

10. The method of claim 1, wherein the light source emits UV light or visible light.

11. The method of claim 10, wherein the light source emits UV light and the gel is formed within five seconds when illuminated with the UV light.

12. The method of claim 10, wherein the light source emits visible light and the gel is formed within ten seconds when the photoinitiator is activated with the visible light.

13. The method of claim 1, wherein the composition further comprises a tyrosine derivative.

14. The method of claim 13, wherein the composition comprises up to 10 mM of the additive.

15. The method of claim 13, wherein the tyrosine derivative comprises tyrosine methyl ester or tyrosine ethyl ester.

16. A method of treating a subject, the method comprising forming a gel on tissue of a gastrointestinal tract of the subject by:
applying to the tissue a first solution comprising:
a macromer comprising a polyethylene glycol (PEG)-based polymer, a poly(ethylenimine)-based polymer, or a poly(1,2-glycerol) carbonate-based polymer, the macromer comprising at least one first functional group, and
a first buffer; and
applying to the tissue a second solution comprising:
a crosslinking agent comprising a second (PEG)-based polymer that comprises a plurality of second functional groups, and
a second buffer, the second buffer having a lower pH than the first buffer;
wherein the first solution contacts the second solution to form the gel on the tissue.

17. The method of claim 16, wherein the at least one first functional group comprises a thiol group or an amine group, and the plurality of second functional groups comprise N-hydroxysuccinimide groups or maleimide groups.

18. The method of claim 16, wherein a molecular weight of the macromer is approximately 2,000 Da and/or a molecular weight of the crosslinking agent is approximately 3,400 Da.

19. The method of claim 16, wherein a molar ratio of the crosslinking agent to the macromer ranges from 3:2 to 7:3.

20. A composition comprising:
a macromer comprising a polyethylene glycol (PEG)-based polymer, a poly(ethylenimine)-based polymer, or a poly(1,2-glycerol) carbonate-based polymer, wherein the macromer comprises at least one thiol group or amine group; and
a crosslinking agent comprising a PEG-based polymer that includes a N-hydroxysuccinimide functional group, a maleimide functional group, or both;
wherein the composition is formulated as a hydrogel, and the hydrogel has a gel strength of at least 2,000 Pa; and
wherein the hydrogel is formulated to withstand a burst pressure of up to approximately 150 mbar when the hydrogel is adhered to colon tissue to fill an aperture in the tissue of about 1 mm by about 5 mm.
